(19) United States
(12) Patent Application Publication
(10) Pub. No.: US 2024/0247071 A2
(43) Pub. Date: Jul. 25, 2024

(54) Compositions and Methods for Enhancing Body Weight and Lean Muscle Mass Using Antagonists Against Leptin Receptor, GDF8 and Activin A

(71) Applicant: Regeneron Pharmaceuticals, Inc., Tarrytown, NY (US)

(72) Inventors: Judith Altarejos, Armonk, NY (US); Jesper Gromada, Concord, MA (US)

(21) Appl. No.: 17/415,589

(22) PCT Filed: Dec. 17, 2019
(86) PCT No.: PCT/US2019/066908

§ 371 Date: Jun. 17, 2021

Related U.S. Application Data
(60) Provisional application No. 62/781,226, filed on Dec. 18, 2018.

(51) Int. Cl. C07K 16/28; A61K 33/26; A61K 39/395; A61K 45/06; A61P 21/00; C07K 16/22

(52) U.S. Cl. CPC C07K 16/2869; A61K 33/26; A61K 39/3955; A61K 45/06; A61P 21/00; C07K 16/22; C07K 2317/56; C07K 2317/565; C07K 2317/76

(57) ABSTRACT

The present invention provides combinations including antagonists of leptin receptor, GDF8 and Activin A and methods of use thereof. Such compositions are effective for example for causing an increase in lean body mass, at least in part, at the expense of fat mass. Methods for treating malnutrition, cachexia and other conditions characterized by insufficient nutrition and weight loss are also provided.

This application is a U.S. National Stage Application filed under 35 U.S.C. § 371 of PCT Application No. PCT/US2019/066908 filed Dec. 17, 2019, which claims priority to U.S. Provisional Application Ser. No. 62/781,226, filed Dec. 18, 2018, each of the applications of which are incorporated by reference in their entireties.

### FIELD OF THE INVENTION

The present invention provides, in part, compositions, including inhibitors of LEPR, GDF8 and Activin A, and methods of treatment for enhancing body weight and lean muscle mass.

### SEQUENCE LISTING

An official copy of the sequence listing is submitted concurrently with the specification electronically via EFS-Web as an ASCII formatted sequence listing with a file name of “10547WOseqlist_ST25.txt”, a creation date of Dec. 17, 2019, and a size of about 26 KB. The sequence listing contained in this ASCII formatted document is part of the specification and is herein incorporated by reference in its entirety.

### BACKGROUND OF THE INVENTION

Growth and differentiation factor-8 (GDF8, also known as myostatin) and Activin A are two important regulators of the development and maintenance of skeletal muscle and muscle mass.

GDF8 is a secreted ligand belonging to the transforming growth factor-β (TGF-β) superfamily of growth factors that acts as a negative regulator of muscle mass. GDF8 antagonists have been used in adult mice with significant positive effects on skeletal muscle mass. A receptor to which GDF8 binds and negatively regulates muscle mass is the activin receptor type IIB (ACVR2B). GDF8 is not the only negative regulator of muscle mass acting via ACVR2B. Activin A (ActA) also negatively regulates muscle mass via this receptor (Latres et al., Activin A more prominently regulates muscle mass in primates than does GDF8, Nature Comm. 8:15153 (2017)). ActRIIB binds ligands including Activin A, B, C and E, GDF11, bone morphogenetic protein 9 (BMP9) and BMP10. Data suggest that Activin A acts in concert with GDF8 to regulate skeletal muscle mass and that combined GDF8 and Activin A inhibition may be effective for the treatment of muscle atrophy in patients with wasting disorders of muscle (Latres et al. (2017)).

Leptin is a polypeptide hormone predominantly expressed by adipose tissue and skeletal muscle and is involved in the regulation of metabolism, energy balance and food intake. Leptin activity is mediated by interaction with, and signaling through, the leptin receptor. Leptin receptor, (also known as “LEPR,” “WSX,” “OB receptor,” “OB-R,” and “CD295”) is a single-pass transmembrane receptor of the class I cytokine receptor family with a large extracellular domain. LEPR regulates body weight via JAK-STAT3 signaling. Altered signaling from leptin or LEPR or both can contribute to multiple disorders including, but not limited to, anorexia or other psychiatric eating disorders, cachexia, autoimmune disorders, cardiovascular diseases and neurodegenerative disorders.

### SUMMARY OF THE INVENTION

The present invention relates to compositions that are useful not only for increasing overall body mass, but also for increasing body mass in a manner which leads to a more desirable overall body composition. As mentioned, administration of antagonistic anti-LEPR, anti-GDF8 and anti-ActA antibodies (triple combination) led to an increase in overall body weight over what was observed with anti-LEPR alone or anti-GDF8 with anti-ActA. Further beneficial effects were observed with respect to the lean mass of subjects who received the triple combination. The overall lean mass increased and the proportion of fat mass-to-lean decreased in these subjects (relative to anti-LEPR alone or anti-GDF8 with anti-ActA). For example, this increase in lean mass was confirmed insofar as subjects receiving the triple combination exhibited greater muscle mass and muscle fiber size (area) in some specific muscle structures assayed.

The present invention provides a combination comprising: a leptin receptor (e.g., human leptin receptor) antagonist in association with a GDF8 (e.g., human GDF8) antagonist in association with an Activin A (e.g., human Activin A) antagonist (e.g., H4H18457P2/H4H1657N2/H4H10446P2); and optionally, a pharmaceutically acceptable carrier. For example, such combinations may include a co-formulation including at least two antagonists selected from leptin receptor antagonist, GDF8 antagonist and Activin A antagonist; or the antagonists can be in separate compositions. The leptin receptor antagonist, GDF8 antagonist and/or Activin A antagonist is, in an embodiment of the invention, an antibody or antigen-binding fragment thereof that binds specifically to leptin receptor, GDF8 and/or Activin A, respectively. In an embodiment of the invention, the leptin receptor antagonist is an antibody or antigen-binding fragment which specifically binds to the receptor and does not compete with leptin for binding to the receptor. In an embodiment of the invention, the LEPR antagonist is an antibody or an antigen-binding fragment thereof that specifically binds to LEPR comprises a heavy chain variable region that comprises CDR-H1, CDR-H2, CDR-H3 of a heavy chain variable region selected from H4H17322P2, H4H18437P2, H4H18439P2, H4H18440P2, H4H18457P2, H4H18462P2, H4H18464P2, H4H18466P2 and H4H18508P2; and CDR-L1, CDR-L2, CDR-L3 of a light chain variable region selected from H4H17322P2, H4H18437P2, H4H18439P2, H4H18440P2, H4H18457P2, H4H18462P2, H4H18464P2, H4H18466P2 and H4H18508P2, or that binds to the same epitope on LEPR as said antibody or fragment and/or that competes for binding to LEPR as said antibody or fragment. In an embodiment of the invention, the GDF8 antagonist is an antibody or an antigen-binding fragment thereof that specifically binds to GDF8 that comprises a heavy chain variable region that comprises CDR-H1, CDR-H2, CDR-H3 of a heavy chain variable region selected from 21-E5; 21-B9; 21-E9; 21-A2; 22-D3; 22-E6; 22-G10; 1A2; 20B12; 58C8; 19F2; 8D12-1; 4E3-7; 9B11-12; 4B9; 1H4-5; 9B4-3; 3E2-1; 4G3-25; 4B6-6; H4H1657N2 or H4H1669P and H4H18508P2; and CDR-L1, CDR-L2, CDR-L3 of a light chain variable region selected from 21-E5; 21-B9; 21-E9; 21-A2; 22-D3; 22-E6; 22-G10; 1A2; 20B12; 58C8; 19F2; 8D12-1; 4E3-7; 9B11-12; 4B9; 1H4-5; 9B4-3; 3E2-1; 4G3-25; 4B6-6; H4H1657N2 or H4H1669P and H4H18508P2, or that binds to the same epitope on GDF8 as said antibody or fragment and/or that competes for binding to GDF8 as said antibody or fragment. In an embodiment of the invention, the Activin A antagonist is an antibody or an antigen-binding fragment thereof that specifically binds to Activin A that comprises a heavy chain variable region that comprises CDR-H1, CDR-H2, CDR-H3 of a heavy chain variable region selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and H2aM10965N and CDR-L1, CDR-L2, CDR-L3 of a light chain variable region selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and H2aM10965N, or that binds to the same epitope on Activin A as said antibody or fragment and/or that competes for binding to Activin A as said antibody or fragment. In an embodiment of the invention, the LEPR antagonist is an antibody or an antigen-binding fragment thereof that comprises a heavy chain variable region selected from H4H17322P2, H4H18437P2, H4H18439P2, H4H18440P2, H4H18457P2, H4H18462P2, H4H18464P2, H4H18466P2 and H4H18508P2; and a light chain variable region selected from H4H17322P2, H4H18437P2, H4H18439P2, H4H18440P2, H4H18457P2, H4H18462P2, H4H18464P2, H4H18466P2 and H4H18508P2, or that binds to the same epitope on LEPR as said antibody or fragment and/or that competes for binding to LEPR as said antibody or fragment. In an embodiment of the invention, the GDF8 antagonist is an antibody or an antigen-binding fragment thereof that comprises a heavy chain variable region selected from 21-E5; 21-B9; 21-E9; 21-A2; 22-D3; 22-E6; 22-G10; 1A2; 20B12; 58C8; 19F2; 8D12-1; 4E3-7; 9B11-12; 4B9; 1H4-5; 9B4-3; 3E2-1; 4G3-25; 4B6-6; H4H1657N2 or H4H1669P and H4H18508P2; and a light chain variable region selected from 21-E5; 21-B9; 21-E9; 21-A2; 22-D3; 22-E6; 22-G10; 1A2; 20B12; 58C8; 19F2; 8D12-1; 4E3-7; 9B11-12; 4B9; 1H4-5; 9B4-3; 3E2-1; 4G3-25; 4B6-6; H4H1657N2 or H4H1669P and H4H18508P2 or that binds to the same epitope on GDF8 as said antibody or fragment and/or that competes for binding to GDF8 as said antibody or fragment. In an embodiment of the invention, the Activin A antagonist is an antibody or an antigen-binding fragment thereof that comprises a heavy chain variable region selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and H2aM10965N; and a light chain variable region selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and H2aM10965N, or that binds to the same epitope on Activin A as said antibody or fragment and/or that competes for binding to Activin A as said antibody or fragment. Such combinations optionally include one or more further therapeutic agents (e.g., an appetite stimulant, a cannabinoid, an angiotensin-converting enzyme (ACE) inhibitor, an angiotensin receptor blocker, a smooth muscle relaxant, a nitrate, a diuretic, iron, a bronchodilator, an anticholinergic, a corticosteroid, an antibiotic, a nonsteroidal anti-inflammatory drug (NSAID), an immunosuppressant, an HMG-CoA reductase inhibitor, an anti-depressant, an anti-cancer therapy and/or a topical agent).

The present invention provides an injection device (e.g., hypodermic needle and syringe, an autoinjector or a pre-filled syringe) or vessel (e.g., a vial) that includes a combination of the present invention (e.g., H4H18457P2/H4H1657N2/H4H10446P2). The present invention further provides methods for administering the combination of the present invention to a subject (e.g., a human) including the step of introducing the components of the combination into the body of the subject, e.g., parenterally, for example, by injection using an injection device according to the present invention. In an embodiment of the invention, the subject suffers from malnutrition, failure to thrive, insufficient food intake, an eating disorder, cachexia, muscle atrophy or wasting and muscle injury and/or is undergoing physical therapy.

The present invention also provides a method for inhibiting LEPR, GDF8 and Activin A in the body of a subject (e.g., a human) comprising administering a therapeutically effective amount of the combination of the present invention (e.g., H4H18457P2/H4H1657N2/H4H10446P2) to the subject e.g., parenterally, for example, by injection using an injection device according to the present invention. In an embodiment of the invention, the subject suffers from malnutrition, failure to thrive, insufficient food intake, an eating disorder, cachexia, muscle atrophy or wasting and muscle injury and/or is undergoing physical therapy.

The present invention also provides a method for increasing food intake, adiposity, body weight, muscle strength, muscle fiber size or lean mass (e.g., at the expense of fat mass), in a subject in need thereof, comprising administering a therapeutically effective amount of the combination of the present invention (e.g., H4H18457P2/H4H1657N2/H4H10446P2) to the subject (e.g., a human). In an embodiment of the invention, the subject suffers from malnutrition, failure to thrive, insufficient food intake, an eating disorder, cachexia, muscle atrophy or wasting and muscle injury and/or is undergoing physical therapy.

The present invention further provides a method for increasing athletic performance in a subject in need thereof (e.g., a human) comprising administering a therapeutically effective amount of the combination of the present invention (e.g., H4H18457P2/H4H1657N2/H4H10446P2) to the subject. In an embodiment of the invention, the subject suffers from malnutrition, failure to thrive, insufficient food intake, an eating disorder, cachexia, muscle atrophy or wasting and muscle injury and/or is undergoing physical therapy (e.g., stroke rehabilitation).

The present invention provides a method for mitigating increased liver triglyceride content in a subject administered an leptin receptor antagonist comprising administering, to the subject, in association with the leptin receptor antagonist, a GDF8 antagonist in association with an Activin A antagonist (e.g., H4H18457P2/H4H1657N2/H4H10446P2).

The present invention also provides a method for treating or preventing malnutrition, cachexia, failure to thrive, an eating disorder characterized by inadequate caloric intake, muscle atrophy, age-related sarcopenia or muscle injury in a subject (e.g., a human) in need thereof comprising administering a therapeutically effective amount of combination of the present invention (e.g., H4H18457P2/H4H1657N2/H4H10446P2) to the subject. For example, in an embodiment of the invention, (i) the subject with malnutrition suffers from hospital malnutrition, failure to thrive in childhood, an eating disorder characterized by inadequate caloric intake, anorexia and/or bulimia; (ii) the subject with cachexia suffers from or is undergoing anorexia bulimia, an eating disorder, a pulmonary disease, chronic obstructive pulmonary disorder (COPD), chronic kidney disease, infectious disease, HIV-infection, acquired immune deficiency syndrome (AIDS), congestive heart failure, radiation treatment, cancer, hepatocellular carcinoma, melanoma, breast cancer, an autoimmune disorder, inflammatory bowel disease, lupus erythematosus, multiple sclerosis, rheumatoid arthritis, Crohn's disease, psoriasis, cystic fibrosis, cardiovascular disease, elevated blood pressure, depression and/or neurodegenerative disorders; and/or (iii) the subject with muscle atrophy or wasting suffers from or is experiencing sepsis, AIDS, renal failure, cardiac failure, excessive glucocorticoids, Cushing syndrome, trauma, muscular disuse, immobilization, bed rest, injury, hip fracture, hip replacement, knee replacement and/or mechanical ventilation.

Methods for making a combination of the present invention (e.g., H4H18457P2/H4H1657N2/H4H10446P2) comprising co-formulating the LEPR antagonist, the GDF8 antagonist and the Activin A antagonist and a pharmaceutically acceptable carrier are also part of the present invention.

The present invention also provides a method of making the device or vessel that comprises a combination of the present invention (e.g., H4H18457P2/H4H1657N2/H4H10446P2) comprising introducing the components of the combination into the vessel or device.

### DETAILED DESCRIPTION OF THE INVENTION

“LEPR/GDF8/ActA” combinations of the present invention include compositions or kits, e.g., pharmaceutical compositions comprising a pharmaceutically acceptable carrier, and one or more LEPR antagonists, one or more GDF8 antagonists and one or more activin A antagonists in association with one another and, optionally, in association with one or more further therapeutic agents. In an embodiment of the invention, the LEPR/GDF8/ActA combination comprises the anti-LEPR antibody H4H18457P2 or an antigen-binding fragment thereof (or a variant thereof), the anti-GDF8 antibody H4H1657N2 or an antigen-binding fragment thereof (or a variant thereof) and the anti-ActA antibody H4H10446P2 or an antigen-binding fragment thereof (or a variant thereof) (H4H18457P2/H4H1657N2/H4H10446P2).

The term “in association with” indicates that the components of the LEPR/GDF8/ActA combinations of the present invention (e.g., H4H18457P2/H4H1657N2/H4H10446P2) are collocated. For example, the LEPR antagonist, the GDF8 antagonist and the Activin A antagonist can be formulated into a single composition, e.g., for simultaneous delivery, or formulated separately into two or more compositions (e.g., and included in a kit). The combination, for example, may be a first composition having the LEPR antagonist co-formulated with the GDF8 antagonist collocated with a second composition which has a separately formulated Activin A antagonist. Alternatively, the combination may be three separately formulated compositions—a first LEPR antagonist composition, a second GDF8 antagonist composition and a third Activin A antagonist composition. Each component of the combination, when formulated separately, can be administered to a subject at a different time than when the other component is administered; for example, each administration may be given non-simultaneously (e.g., separately or sequentially) at intervals over a given period of time in a treatment regimen. Moreover, the separate components may be administered to a subject by the same or by a different route.

Anti-LEPR, anti-GDF8 and/or anti-ActA antibodies or antigen-binding fragments thereof, whose sequences appear in prior publications, are referred to herein. In an embodiment of the invention, such antibodies or fragments comprise at least one heavy chain variable domain (V_{H}) and/or at least one light chain variable region (V_{L}) whose sequence is referred to herein except that each may independently have 1, 2, 3, 4, 5, 6, 7, 8, 9 or 10 point mutations and/or point deletions. Anti-LEPR, anti-GDF8 and/or anti-ActA antibodies or fragments may include the heavy chain CDRs of V_{H}s (HCDRs) whose sequences are referred to herein except that each may independently have 1, 2, 3, 4, 5, 6, 7, 8, 9 or 10 point mutations and/or point deletions; and/or anti-LEPR, anti-GDF8 and/or anti-ActA antibodies or fragments may include the light chain CDRs of V_{L}s (HCDRs) whose sequences are referred to herein except that each may independently have 1, 2, 3, 4, 5, 6, 7, 8, 9 or 10 point mutations and/or point deletions. Antibodies or fragments including such mutations may be referred to herein as “variants”. A “variant” of a polypeptide (e.g., V_{L}, V_{H}, HCDRs or LCDRs) may comprise a sequence having at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identity or similarity to a referenced sequence; for example when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

Sequence identity refers to the degree to which the amino acids of two polypeptides are the same at equivalent positions when the two sequences are optimally aligned. Sequence similarity includes identical residues and nonidentical, biochemically related amino acids. Biochemically related amino acids that share similar properties and may be interchangeable are discussed above.

A leptin receptor (LEPR, OB receptor, OB-R, CD295 or WSX) is, in an embodiment of the invention, the human leptin receptor, comprising the amino acid sequence as set forth in UniProtKB/Swiss-Prot Accession No. P48357.

GDF8 (growth and differentiation factor-8, MSTN or myostatin) includes a protein having the amino acid sequence set forth under UniProtKB/Swiss-Prot accession no. O14793.

Activins are homo- and hetero-dimeric molecules comprising beta subunits, i.e., Inhibin βΔ, inhibin βB, inhibin βθ, and/or inhibin βE. Activin A is a homodimer of two βA subunits; Activin B is a homodimer of two βB subunits; Activin AB is a heterodimer of one βA subunit and one βB subunit; and Activin AC is a heterodimer of one βA subunit and one βθ subunit. In an embodiment of the invention, the inhibin beta A chain amino acid sequence is set forth in under UniProtKB/Swiss-Prot accession no. P08476.

Lean mass is, in an embodiment of the invention, as determined using micro-computed tomography (μCT) or dual-energy X-ray absorptiometry (DXA).

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3.sup.rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399). Single chain antibodies and diabodies are described (see, e.g., Malecki et al. (2002) Proc. Natl. Acad. Sci. USA 99:213-218; Conrath et al. (2001) J. Biol. Chem. 276:7346-7350; Desmyter et al. (2001) J. Biol. Chem. 276:26285-26290; Hudson and Kortt (1999) J. Immunol. Methods 231:177-189; and U.S. Pat. No. 4,946,778). Bifunctional antibodies are provided (see, e.g., Mack, et al. (1995) Proc. Natl. Acad. Sci. USA 92:7021-7025; Carter (2001) J. Immunol. Methods 248:7-15; Volkel, et al. (2001) Protein Engineering 14:815-823; Segal, et al. (2001) J. Immunol. Methods 248:1-6; Brennan, et al. (1985) Science 229:81-83; Raso, et al. (1997) J. Biol. Chem. 272:27623; Morrison (1985) Science 229:1202-1207; Traunecker, et al. (1991) EMBO J. 10:3655-3659; and U.S. Pat. Nos. 5,932,448, 5,532,210, and 6, 129,914). Fully human antibodies may also be developed in genetically engineered mice such as the VelociMouse. See e.g., DeChiara et al., Producing fully ES cell-derived mice from eight-cell stage embryo injections, Methods Enzymol, 476:285-94 (2010); Dechiara et al., VelociMouse: fully ES cell-derived FO-generation mice obtained from the injection of ES cells into eight-cell-stage embryos. Methods Mol Biol, 530:311-24 (2009); U.S. Pat. Nos. 7,576,259; 7,659,442; or 7294754, and US2008/0078000A1.

Purification of antigen is not typically necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2.sup.nd ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### Leptin Receptor Antagonists

The present invention includes LEPR/GDF8/ActA combinations (e.g., H4H18457P2/H4H1657N2/H4H10446P2) which include one or more LEPR antagonists. In an embodiment of the invention, a LEPR antagonist is an anti-LEPR antibody or antigen-binding fragment thereof that does not compete with leptin for binding to LEPR.

LEPR antagonists include antibodies and antigen-binding fragments thereof and other substances (e.g., peptides and small molecules) that specifically bind to LEPR and antagonize one or more biological activities of LEPR. Molecules that specifically bind to LEPR may be referred to as “anti-LEPR”. In an embodiment of the invention, a LEPR antagonist inhibits LEPR signaling, e.g., by binding human LEPR and antagonizing activation of the LEPR-dependent intracellular signaling cascade. Antagonism of LEPR may, in an embodiment of the invention, be achieved by blocking LEPR/leptin binding, e.g., by formation of a complex between the antagonist and leptin or LEPR or both. LEPR signaling antagonism includes, for example, reduction of LEPR-dependent transcriptional activation of STAT3. See e.g., Villanueva & Myers, Int. J. Obes. 32(Suppl 7): S8-12 (2008) and Park & Ahima, F1000Prime Reports 6:73 (2014).

In an embodiment of the invention, a LEPR antagonist is a mutant version of leptin, e.g., a PEGylated mutant leptin. For example, in an embodiment of the invention, the LEPR antagonist is a mammalian (e.g., human) leptin polypeptide in which the LDFI hydrophobic binding site is modified such that from two to four amino acid residues of said hydrophobic binding site are substituted with different amino acid residues such that the site becomes less hydrophobic, said modified, mammalian leptin polypeptide being a leptin antagonist; or a fragment of said modified mammalian leptin polypeptide comprising said altered hydrophobic binding site, wherein said fragment is itself a leptin antagonist. For example, wherein two or more amino acids of the LDFI motif are substituted with alanine, arginine, aspartic acid, glutamic acid, glycine, lysine or serine, e.g., having the mutations L39A/D40A/F41A/142A. Such a leptin mutant may be PEGylated, e.g., with one or more 4000-6000 dalton PEG molecules. See U.S. Pat. No. 7,307,142.

In an embodiment of the invention, the LEPR antagonist is an antibody or antigen-binding fragment selected from H4H17322P2, H4H18437P2, H4H18439P2, H4H18440P2, H4H18457P2, H4H18462P2, H4H18464P2, H4H18466P2 and H4H18508P2; or any other antibody or antigen-binding fragment set forth in WO2018/089532.

In an embodiment of the invention, the anti-LEPR antibody is H4H18457P2.

In an embodiment of the invention, the anti-LEPR antibody or fragment of the present invention comprises:
(i) the HCDRs (HCDR1, HCDR2 and HCDR3) of a heavy chain variable region (or having variants of one or more of the HCDRs) and/or the LCDRs (LCDR1, LCDR2 and LCDR3) of a light chain variable region (or having variants of one or more of the LCDRs) of an antibody selected from H4H17322P2, H4H18437P2, H4H18439P2, H4H18440P2, H4H18457P2, H4H18462P2, H4H18464P2, H4H18466P2 and H4H18508P2; and/or
(ii) the heavy chain variable region (or a variant thereof) and/or the light chain variable region (or a variant thereof) of an antibody selected from H4H17322P2, H4H18437P2, H4H18439P2, H4H18440P2, H4H18457P2, H4H18462P2, H4H18464P2, H4H18466P2 and H4H18508P2;
   and/or is characterized as:
(iii) competing for binding to LEPR (e.g., LEPR with a C-terminal myc-myc-His6 tag) with H4H17322P2, H4H18437P2, H4H18439P2, H4H18440P2, H4H18457P2, H4H18462P2, H4H18464P2, H4H18466P2 and/or H4H18508P2;
   and/or
(iv) binding to LEPR at the same epitope as H4H17322P2, H4H18437P2, H4H18439P2, H4H18440P2, H4H18457P2, H4H18462P2, H4H18464P2, H4H18466P2 and/or H4H18508P2, e.g., wherein the epitope is the extracellular domain of LEPR, the LEPR CRH (cytokine receptor homology) domain 2, the CRH2 domain, the FNIII (fibronectin type III) domain or the Ig(D3) domain.

See international patent application publication no. WO2018/89532.

In an embodiment of the invention, such an antibody or fragment comprises a heavy chain constant domain selected from IgG1, IgG2, IgG3 and IgG4 and/or a light chain constant domain selected from kappa and lambda.

In an embodiment of the invention, the LEPR antagonist is the antibody H4H18457P2 or an antigen-binding fragment thereof.

In an embodiment of the invention the LEPR antagonist is an antibody or antigen-binding fragment thereof comprising:
(1)

a V_{H} comprising the amino acid sequence:
EVQLVESGGSVVRPGESLRLSCAASGFTFDDYGMSWVRQAPGKGLEWVSG
ISWNGGITVYADSVKGRFTVSRDNAKNSLYLQMNSLRAEDTALYHCARAR
YGGADYWGQGTLVTVSS (SEQ ID NO: 1; or a variant
thereof)
and/or
a V_{L} comprising the amino acid sequence:
DIQMTQSPSSLSASVGDRVTITCRASQSISSYLNWYQQKPGKAPKLLIYA
ASSLQSGVPSRFSGSGSGTDETLTISSLQPEDFATYYCQQSYSTPPITFG
QGTRLEIK (SEQ ID NO: 2; or a variant thereof);

and/or
(2)
   a V_{L} comprising the CDR-Ls thereof, e.g.:
   CDR-L1 comprising the amino acid sequence: Gln Ser Ile Ser Ser Tyr (SEQ ID NO: 3; or a variant thereof);
   CDR-L2 comprising the amino acid sequence: Ala Ala Ser (SEQ ID NO: 4; or a variant thereof); and
   CDR-L3 comprising the amino acid sequence: Gln Gln Ser Tyr Ser Thr Pro Pro Ile Thr (SEQ ID NO: 5; or a variant thereof); and/or
   a V_{H} comprising the CDR-Hs thereof, e.g.:
   CDR-H1 comprising the amino acid sequence: Gly Phe Thr Phe Asp Asp Tyr Gly (SEQ ID NO: 6; or a variant thereof);
   CDR-H2 comprising the amino acid sequence: Ile Ser Trp Asn Gly Gly Ile Thr (SEQ ID NO: 7; or a variant thereof); and
   CDR-H3 comprising the amino acid sequence: Ala Arg Ala Arg Tyr Gly Gly Ala Asp Tyr (SEQ ID NO: 8; or a variant thereof);
   and/or
(3)

a heavy chain immunoglobulin comprising the amino
acid sequence:
(SEQ ID NO: 25)
EVQLVESGGSVVRPGESLRLSCAASGFTFDDYGMSWVRQAPGKGLEWVSG
ISWNGGITVYADSVKGRFTVSRDNAKNSLYLQMNSLRAEDTALYHCARAR
YGGADYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK (or
a variant thereof)
and
a light chain immunoglobulin comprising the amino
acid sequence:
(SEQ ID NO: 26)
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC (or a variant thereof).

In an embodiment of the invention, such a V_{L} is linked to a human kappa or lambda light chain immunoglobulin constant domain and/or such a V_{H} is linked to a human IgG (e.g., IgG1, IgG2, IgG3 or IgG4 (for example, an S228P mutant IgG4)) heavy chain immunoglobulin constant domain.

### GDF8 Antagonists

The present invention includes LEPR/GDF8/ActA combinations (e.g., H4H18457P2/H4H1657N2/H4H10446P2) which include one or more GDF8 antagonists.

GDF8 antagonists include antibodies and antigen-binding fragments thereof and other substances (e.g., peptides and small molecules) that specifically bind to GDF8 and antagonize one or more biological activities of GDF8. Molecules that specifically bind to GDF8 may be referred to as “anti-GDF8”. For example, in an embodiment of the invention, the antagonist blocks interaction between GDF8 and Activin RIIB (or an Fc fusion thereof) or inhibits SMAD-dependent activation of A204 cells stably expressing Smad-dependent (CAGA12) luciferase.

In an embodiment of the invention, a GDF8 antagonist is a small molecule such as dorsomorphin (Millipore Sigma; St. Louis, MO) or LDN-193189 (Millipore Sigma; St. Louis, MO).

In an embodiment of the invention, a GDF8 antagonist specifically binds GDF8 but, for example, does not bind other ActRIIB ligands such as GDF3, BMP2, BMP4, BMP7, BMP9, BMP10, GDF11, Activin A, Activin B, Activin AB, and/or Nodal.

In an embodiment of the invention, the GDF8 antagonist is an anti-GDF8 antibody or antigen-binding fragment thereof. Anti-GDF8 antibodies are mentioned in, e.g., U.S. Pat. Nos. 6,096,506; 7,320,789; 7,261,893; 7,807,159; 7,888,486; 7,635,760; 7,632,499; in U.S. Patent Appl. Publ. Nos. 2007/0178095; 2010/0166764; and 2009/0148436; and International Patent Appl. Publ. No. WO2010/070094.

Anti-GDF8 antibodies are also described in U.S. patent application Ser. No. 13/115,170, filed on May 25, 2011, and published as US2011/0293630, now U.S. Pat. No. 8,840,894 or International Patent Application No. PCT/US2012/064911, filed Nov. 14, 2012 (WO2013/074557), including the antibodies designated 21-E5; 21-B9; 21-E9; 21-A2; 22-D3; 22-E6; 22-G10; 1A2; 20B12; 58C8; 19F2; 8D12-1 (or 8D12); 4E3-7; 9B11-12; 4B9; 1H4-5; 9B4-3; 3E2-1; 4G3-25; 4B6-6; H4H1657N2 or H4H1669P or an antigen-binding fragment thereof.

In an embodiment of the invention, the GDF8 antagonist is an anti-GDF8 antibody or antigen-binding fragment selected from 21-E5; 21-B9; 21-E9; 21-A2; 22-D3; 22-E6; 22-G10; 1A2; 20B12; 58C8; 19F2; 8D12-1 (or 8D12); 4E3-7; 9B11-12; 4B9; 1H4-5; 9B4-3; 3E2-1; 4G3-25; 4B6-6; H4H1657N2 or H4H1669P; or any anti-GDF8 antibody or antigen-binding fragment set forth in US2011/0293630.

In an embodiment of the invention, the anti-GDF8 antibody is H4H1657N2.

In an embodiment of the invention, the anti-GDF8 antibody or fragment of the present invention comprises:
(i) the HCDRs (HCDR1, HCDR2 and HCDR3) of a heavy chain variable region (or having variants of one or more of the HCDRs) and/or the LCDRs (LCDR1, LCDR2 and LCDR3) of a light chain variable region (or having variants of one or more of the LCDRs) of an antibody selected from 21-E5; 21-B9; 21-E9; 21-A2; 22-D3; 22-E6; 22-G10; 1A2; 20B12; 58C8; 19F2; 8D12-1 (or 8D12); 4E3-7; 9B11-12; 4B9; 1H4-5; 9B4-3; 3E2-1; 4G3-25; 4B6-6; H4H1657N2 or H4H1669P; and/or
(ii) the heavy chain variable region (or a variant thereof) and/or light chain variable region (or a variant thereof) of an antibody selected from 21-E5; 21-B9; 21-E9; 21-A2; 22-D3; 22-E6; 22-G10; 1A2; 20B12; 58C8; 19F2; 8D12-1 (or 8D12); 4E3-7; 9B11-12; 4B9; 1H4-5; 9B4-3; 3E2-1; 4G3-25; 4B6-6; H4H1657N2 or H4H1669P;
   and/or is characterized as:
(iii) competing for binding to GDF8 with 21-E5; 21-B9; 21-E9; 21-A2; 22-D3; 22-E6; 22-G10; 1A2; 20B12; 58C8; 19F2; 8D12-1 (or 8D12); 4E3-7; 9B11-12; 4B9; 1H4-5; 9B4-3; 3E2-1; 4G3-25; 4B6-6; H4H1657N2 or H4H1669P;
   and/or
(iv) binding to GDF8 at the same epitope as 21-E5; 21-B9; 21-E9; 21-A2; 22-D3; 22-E6; 22-G10; 1A2; 20B12; 58C8; 19F2; 8D12-1 (or 8D12); 4E3-7; 9B11-12; 4B9; 1H4-5; 9B4-3; 3E2-1; 4G3-25; 4B6-6; H4H1657N2 or H4H1669P, e.g., wherein the epitope is GDF8 amino acids 1-14, 48-65, 48-69, 48-72, 52-65, 52-72, 56-65, 56-72 and/or 73-90; or a peptide consisting of such amino acids (e.g., comprising a C-terminal tag such as biotin).

See published US patent application no. US2011/0293630. In an embodiment of the invention, such an antibody or fragment comprises a heavy chain constant domain selected from IgG1, IgG2, IgG3 and IgG4 and/or a light chain constant domain selected from kappa and lambda.

Antibody H4H1657N2 may be referred to as REGN1033 or trevogrumab.

In an embodiment of the invention the GDF8 antagonist is an antibody or antigen-binding fragment thereof comprising:
(1)

a V_{H} comprising the amino acid sequence:
EVQVLESGGDLVQPGGSLRLSCAASGFTFSAYAMTWVRQAPGKGLEWVSA
ISGSGGSAYYADSVKGRFTISRDNSKNTVYLQMNSLRAEDTAVYYCAKDG
AWKMSGLDVWGQGTTVIVSS (SEQ ID NO: 9; or a variant
thereof);
and
a V_{L} comprising the amino acid sequence:
DIQMTQSPASLSASVGDRVTITCRASQDISDYLAWYQQKPGKIPRLLIYT
TSTLQSGVPSRFRGSGSGTDFTLTISSLQPEDVATYYCQKYDSAPLTFGG
GTKVEIK (SEQ ID NO: 10; or a variant thereof)

and/or
(2)
   a V_{L} comprising the CDR-Ls thereof, e.g.:
   CDR-L1 comprising the amino acid sequence: Gln Asp Ile Ser Asp Tyr (SEQ ID NO: 11; or a variant thereof)
   CDR-L2 comprising the amino acid sequence: Thr Thr Ser (SEQ ID NO: 12; or a variant thereof)
   CDR-L3 comprising the amino acid sequence: Gln Lys Tyr Asp Ser Ala Pro Leu Thr (SEQ ID NO: 13; or a variant thereof)
   and/or
   a V_{H} comprising the CDR-Hs thereof, e.g.:
   CDR-H1 comprising the amino acid sequence: Gly Phe Thr Phe Ser Ala Tyr Ala (SEQ ID NO: 14; or a variant thereof)
   CDR-H2 comprising the amino acid sequence: Ile Ser Gly Ser Gly Gly Ser Ala (SEQ ID NO: 15; or a variant thereof)
   CDR-H3 comprising the amino acid sequence: Ala Lys Asp Gly Ala Trp Lys Met Ser Gly Leu Asp Val (SEQ ID NO: 16; or a variant thereof); and/or
(3)

a heavy chain immunoglobulin comprising the amino
acid sequence:
(SEQ ID NO 27)
EVQVLESGGDLVQPGGSLRLSCAASGFTFSAYAMTWVRQAPGKGLEWVSA
ISGSGGSAYYADSVKGRFTISRDNSKNTVYLQMNSLRAEDTAVYYCAKDG
AWKMSGLDVWGQGTTVIVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
(or a variant thereof),
and
a light chain immunoglobulin comprising the amino
acid sequence:
(SEQ ID NO 28)
DIQMTQSPASLSASVGDRVTITCRASQDISDYLAWYQQKPGKIPRLLIYT
TSTLQSGVPSRFRGSGSGTDFTLTISSLQPEDVATYYCQKYDSAPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC (or a variant thereof)

In an embodiment of the invention, such a V_{L} is linked to a human kappa or lambda light chain immunoglobulin constant domain and/or such a V_{H} is linked to a human IgG (e.g., IgG1, IgG2, IgG3 or IgG4 (for example, an S228P mutant IgG4)) heavy chain immunoglobulin constant domain.

### Activin A Antagonists

The present invention includes LEPR/GDF8/ActA combinations (e.g., H4H18457P2/H4H1657N2/H4H10446P2) which include one or more Activin A antagonists.

Activin A antagonists include antibodies and antigen-binding fragments thereof and other substances (e.g., peptides and small molecules) that specifically bind to Activin A and antagonize one or more biological activities of Activin A. Molecules that specifically bind to Activin A may be referred to as “anti-Activin A” or “anti-ActA”. In an embodiment of the invention, such an ActA antagonist:
(i) interferes with the interaction between Activin A and an Activin A receptor (e.g., Activin Type IIA receptor, Activin Type IIB receptor, Activin Type I receptor, etc.);
(ii) interferes with the formation of Activin-Activin receptor complexes; and/or
(iii) results in inhibition of at least one biological function of Activin A such as phosphorylation and activation of Type I Activin receptors and the phosphorylation of SMAD2 and 3 proteins.

Activin A antagonists, such as antibodies and antigen-binding fragments thereof and other substances (e.g., peptides) specifically bind to Activin A or the βA subunit thereof. An antigen-specific binding protein that specifically binds the βA subunit may recognize both Activin A (βA/βA homodimer) and Activin AB (βA/βB heterodimer). In an embodiment of the invention, an Activin A-specific binding protein binds both Activin A and Activin AB (but not Activin B). Anti-Activin A antibodies and antigen-binding fragments are mentioned in, e.g., US2009/0234106. A particular anti-Activin A antibody is designated “MAB3381,” and is available commercially from R&D Systems, Inc, Minneapolis, MN. MAB3381 specifically binds Activin A (homodimer) as well as Activin AB (heterodimer).

In an embodiment of the invention, the Activin A antagonist is an antibody or antigen-binding fragment selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and H2aM10965N; or any other antibody or antigen-binding fragment set forth in WO2015/017576.

In an embodiment of the invention, the anti-Activin A antibody is garetosmab.

In an embodiment of the invention, the anti-Activin A antibody or fragment of the present invention comprises:
(i) the HCDRs (HCDR1, HCDR2 and HCDR3) of a heavy chain variable region (or having variants of one or more of the HCDRs) and/or the LCDRs (LCDR1, LCDR2 and LCDR3) of a light chain variable region (or having variants of one or more of the LCDRs) of an antibody selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and H2aM10965N; and/or
(ii) the heavy chain variable region (or a variant thereof) and/or light chain variable region (or a variant thereof) of an antibody selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2,
H4H10445P2, H4H10446P22, H4H10447P2, H4H10448P2, H4H10452P2, H4H10468P2 or H2aM10965N;
   and/or is characterized as:
(iii) competing for binding to Activin A with H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and/or H2aM10965N;
   and/or
(iv) binding to Activin A at the same epitope as H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and/or H2aM10965N.

See published international patent application publication no. WO2015/017576. In an embodiment of the invention, such an antibody or fragment comprises a heavy chain constant domain selected from IgG1, IgG2, IgG3 and IgG4 and/or a light chain constant domain selected from kappa and lambda.

Antibody H4H10446P2 may be referred to as REGN2477 or garetosmab.

In an embodiment of the invention the Activin A antagonist is an antibody or antigen-binding fragment thereof comprising:
(1)

a V_{H} comprising the amino acid sequence:
QVQLQESGPGLVKPSETLSLTCTVSGGSFSSHFWSWIRQPPGKGLEWIGY
ILYTGGTSFNPSLKSRVSMSVGTSKNQFSLKLSSVTAADTAVYYCARARS
GITFTGIIVPGSFDIWGQGTMVTVSS (SEQ ID NO: 17; or a
variant thereof);
and
a V_{L} comprising the amino acid sequence:
EIVLTQSPGTLSLSPGERATLSCRASQSVSSSYLAWYQQKPGQAPRLLIY
GASSRATGIPDRESGSGSGTDFTLTISRLEPEDFAVYYCQQYGSSPWTFG
QGTKVEIK (SEQ ID NO: 18; or a variant thereof);

and/or
(2)
   a V_{L} comprising the CDR-Ls thereof, e.g.:
   CDR-L1 comprising the amino acid sequence: Gln Ser Val Ser Ser Ser Tyr (SEQ ID NO: 19; or a variant thereof);
   CDR-L2 comprising the amino acid sequence: Gly Ala Ser (SEQ ID NO: 20; or a variant thereof); and
   CDR-L3 comprising the amino acid sequence: Gln Gln Tyr Gly Ser Ser Pro Trp Thr (SEQ ID NO: 21; or a variant thereof);
   and
   a V_{H} comprising the CDR-Hs thereof, e.g.:
   CDR-H1 comprising the amino acid sequence: Gly Gly Ser Phe Ser Ser His Phe (SEQ ID NO: 22; or a variant thereof);
   CDR-H2 comprising the amino acid sequence: Ile Leu Tyr Thr Gly Gly Thr (SEQ ID NO: 23; or a variant thereof); and
   CDR-H3 comprising the amino acid sequence: Ala Arg Ala Arg Ser Gly Ile Thr Phe Thr Gly Ile Ile Val Pro Gly Ser Phe Asp Ile (SEQ ID NO: 24; or a variant thereof);
   and/or
(3)

a heavy chain immunoglobulin comprising the amino
acid sequence:
(SEQ ID NO: 29)
QVQLQESGPGLVKPSETLSLTCTVSGGSFSSHFWSWIRQPPGKGLEWIGY
ILYTGGTSFNPSLKSRVSMSVGTSKNQFSLKLSSVTAADTAVYYCARARS
GITFTGIIVPGSFDIWGQGTMVTVSSASTKGPSVFPLAPCSRSTSESTAA
LGCLVKDYFPEPVTVSWNSGALTSGVHTFPAVLQSSGLYSLSSVVTVPSS
SLGTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLS
LGK (or a variant thereof)
and
a light chain immunoglobulin comprising the amino
acid sequence:
(SEQ ID NO: 30)
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC (or a variant thereof).

In an embodiment of the invention, such a V_{L} is linked to a human kappa or lambda light chain immunoglobulin constant domain and/or such a V_{H} is linked to a human IgG (e.g., IgG1, IgG2, IgG3 or IgG4 (for example, an S228P mutant IgG4)) heavy chain immunoglobulin constant domain.

### Pharmaceutical Compositions

The present invention includes pharmaceutical formulations of the LEPR/GDF8/ActA combinations of the present invention (e.g., H4H18457P2/H4H1657N2/H4H10446P2), including, for example, or one or more (e.g., 3) components thereof admixed with a pharmaceutically acceptable carrier or excipient. See, e.g., Remington's Pharmaceutical Sciences and U.S. Pharmacopeia: National Formulary, Mack Publishing Company, Easton, Pa. (1984). Methods for making such a pharmaceutical formulation comprising admixing a pharmaceutically acceptable carrier or excipient with the component(s) forms part of the present invention as do the pharmaceutical compositions that are produced by such methods.

The scope of the present invention includes desiccated, e.g., freeze-dried, LEPR/GDF8/ActA combinations of the present invention (e.g., H4H18457P2/H4H1657N2/H4H10446P2) or one or more components thereof or a pharmaceutical composition thereof that includes a pharmaceutically acceptable carrier but substantially lacks water. In an embodiment of the invention, the pharmaceutical formulation is aqueous (includes water). In an embodiment of the invention, the pharmaceutical formulation is sterile.

Pharmaceutical formulations of therapeutic agents may be prepared by mixing with acceptable carriers, excipients, or stabilizers in the form of, e.g., lyophilized powders, slurries, aqueous solutions or suspensions (see, e.g., Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

The mode of administration of LEPR/GDF8/ActA combinations can vary. Routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

The present invention provided methods for administering pharmaceutical formulations comprising a LEPR/GDF8/ActA combination (e.g., H4H18457P2/H4H1657N2/H4H10446P2) to a subject (e.g., a human) comprising introducing the formulation into the body of the subject, e.g., into a vein, the subcutis or the muscular tissue of the subject. For example, the method comprises piercing the body of the subject with a needle of a syringe and injecting the formulation into the body of the subject. Such a method includes introducing a formulation comprising all three components of the combination, which are co-formulated, in the body of the subject; or, for example, introducing three separately formulated components of the combination in the body of the subject.

The present invention provides one or more vessels (e.g., a plastic or glass vial, e.g., with a cap, or a chromatography column, hollow bore needle or a syringe cylinder) comprising LEPR/GDF8/ActA combinations of the present invention (e.g., H4H18457P2/H4H1657N2/H4H10446P2) or a pharmaceutical composition thereof comprising a pharmaceutically acceptable carrier. The present invention includes methods for preparing one or more vessels comprising the combination comprising introducing the components of the combination into one or more vessels, e.g., a single vessel comprising a combination of components which are co-formulated. In an embodiment of the invention, the vessel(s) is/are then introduced into a kit.

The present invention also provides a device, e.g., an injection device, comprising LEPR/GDF8/ActA combinations of the present invention (e.g., H4H18457P2/H4H1657N2/H4H10446P2) or a pharmaceutical composition thereof and methods of use thereof. An injection device is a device that introduces a substance into the body of a patient via a parenteral route, e.g., intramuscular, subcutaneous or intravenous. For example, an injection device may be a syringe (e.g., pre-filled with the pharmaceutical composition, such as an auto-injector, or filled at the point of use, e.g., by the user or a clinician) which, for example, includes a cylinder or barrel for holding fluid to be injected (e.g., comprising the antibody or fragment or a pharmaceutical composition thereof), a needle for piercing skin and/or blood vessels for injection of the fluid; and a plunger for pushing the fluid out of the cylinder and through the needle bore.

The pharmaceutical compositions disclosed herein may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556. Such needleless devices and methods of use thereof comprising the pharmaceutical composition are also part of the present invention.

The present invention includes methods for preparing one or more injection devices (e.g., pre-filled syringe or autoinjector) comprising the LEPR/GDF8/ActA combination (e.g., H4H18457P2/H4H1657N2/H4H10446P2) comprising introducing the components of the combination into one or more of such devices, e.g., a single device comprising the combination components which are co-formulated. In an embodiment of the invention, the injection device(s) is/are then introduced into a kit.

The present invention also includes kits comprising the a LEPR/GDF8/ActA combination of the invention (e.g., H4H18457P2/H4H1657N2/H4H10446P2). In an embodiment of the invention, the kit comprises each antagonist in a separate vessel or injection device (e.g., pre-filled syringe or autoinjector); or all three antagonists co-formulated in a single vessel or injection device. The kit can include a package insert including information concerning the pharmaceutical compositions and dosage forms in the kit. Generally, such information aids patients and physicians in using the enclosed pharmaceutical compositions effectively and safely. For example, any of the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### Treatment and Administration

The LEPR/GDF8/ActA combination has demonstrated an exceptional ability to bring about an increase in lean mass in subjects administered the combination. Such increases in lean mass possibly is at the expense of increases in fat mass. The LEPR/GDF8/ActA combination brings about greater increases in lean mass vs blockade of only GDF8 and ActA and there is less of an increase in fat mass as compared to what is observed with the LEPR antagonist alone. This may be due to calories from the fat now being used to build the extra muscle.

Methods for administering a LEPR/GDF8/ActA combination of the present invention (e.g., H4H18457P2/H4H1657N2/H4H10446P2) to a subject include introducing components of the combination, in association with one another, into the body of the subject. Such introduction may be done by any acceptable route, e.g., parenterally. Such components may be co-formulated into a single composition or formulated into separate compositions. Administration of one or more of the components in separate compositions may be separated in time if such administrations are done as part of a regimen. In an embodiment of the invention, the method comprises injecting all three components into the subject at once; in another embodiment of the invention, the GDF8 antagonist and the ActA antagonist are formulated together and are injected at once along with the LEPR antagonist in a separate injection; in another embodiment of the invention, all three antagonists are injected separately in three individual injections (e.g., subcutaneous, intravenous or intramuscular or a combination of two or three of such routes).

The scope of the present invention provides methods for increasing food intake, adiposity, body weight (e.g., at the expense of fat mass), muscle strength, muscle fiber size or lean mass (e.g., at the expense of fat mass) in a subject in need thereof comprising administering a therapeutically effective amount of LEPR/GDF8/ActA combinations (e.g., H4H18457P2/H4H1657N2/H4H10446P2) to the subject.

Moreover, LEPR/GDF8/ActA combinations of the present invention (e.g., H4H18457P2/H4H1657N2/H4H10446P2) may be used to:
treat or prevent a disease or condition which would be cured or ameliorated to any degree by antagonism of (i) LEPR (e.g., hyperleptinemia or elevated expression of the OB-R leptin receptor that results in excess LEPR signaling), (ii) GDF8 (e.g., muscle atrophy/wasting) and/or (iii) ActA; and/or
treat or prevent a disease or condition which would be cured or ameliorated to any degree by causing an increase or reversing decreases in food intake, adiposity, body weight (e.g., at the expense of fat mass), muscle fiber size muscle strength or lean mass (e.g., at the expense of fat mass);
by administering a therapeutically effective amount of LEPR/GDF8/ActA combinations (e.g., H4H18457P2/H4H1657N2/H4H10446P2) to the subject. Such diseases or conditions that can be treated or prevented include, for example, malnutrition, failure to thrive, insufficient food/caloric intake, eating disorders, cachexia, muscle atrophy/wasting, age-related sarcopenia, and muscle injury. Such conditions and diseases are discussed in detail herein.

Malnutrition is an example of a condition, in a subject, that would benefit from LEPR/GDF8/ActA combination therapy. Malnutrition is a term used to describe any imbalance in nutrition; from over-nutrition to under-nutrition e.g., as seen in hospitals and residential care facilities. For the purposes of the present invention, malnutrition refers to under-nutrition (unless otherwise stated). Thus, the present invention provides methods for treating or preventing malnutrition in a subject, e.g., who is in a hospital or residential care facility, by administering a therapeutically effective amount of LEPR/GDF8/ActA combination to the subject in need thereof.

Malnutrition can develop as a consequence of:
deficiency in dietary intake;
increased requirements associated with a disease state (e.g., HIV-infected subjects and subjects with AIDS (acquired immune deficiency syndrome) or cystic fibrosis may require increased dietary intake in order to maintain normal body weight) for which an increase in dietary intake is not sufficient to compensate; and/or
complications of an underlying illness (e.g., patients suffering from cirrhosis, chronic pancreatitis, lactase deficiency, pancreatic cancer, amyloidosis, celiac disease, Crohn's disease, radiation enteritis and Addison's disease may suffer from malabsorption due to insufficient digestive agents; and patients suffering from cancer or infectious diseases (or other diseases) may develop cachexia secondary to the underlying illness (see below)) for which an increase in dietary intake is not sufficient to compensate.

Thus, the present invention includes methods for reversing or halting malnutrition in a subject which is the consequence of a deficiency in dietary intake, increased requirements associated with a disease state (e.g., HIV or AIDS), and/or complications of an underlying illness comprising administering a therapeutically effective amount of LEPR/GDF8/ActA combination (e.g., H4H18457P2/H4H1657N2/H4H10446P2) to the subject.

Malnutrition is associated with negative outcomes for patients, including higher infection and complication rates, increased muscle loss, impaired wound healing, longer length of hospital stay and increased morbidity and mortality. Thus, the present invention includes methods for reducing negative outcomes (e.g., for reducing the possibility of infection or for preventing impaired wound healing) for a subject which are associated with malnutrition comprising administering a therapeutically effective amount of LEPR/GDF8/ActA combinations (e.g., H4H18457P2/H4H1657N2/H4H10446P2) to the subject.

Numerous nutrition screening and assessment tools exist to identify the risk of malnutrition and to diagnose malnutrition. For example, The Malnutrition Screening Tool (MST) is a simple, three-question tool assessing recent weight and appetite loss validated for use in general medical, surgical and oncology patients (Anthony, Nutrition screening tools for hospitalized patients. Nutr. Clin. Pract. 2008; 23:373-382; Ferguson et al., Validation of a malnutrition screening tool for patients receiving radiotherapy. Australas. Radiol. 1999; 43:325-327). The Mini Nutrition Assessment (MNA) was developed specifically for use among elderly patients (≥65 years) in hospitals, nursing homes and the community and is thus limited to this demographic (Anthony, Nutrition screening tools for hospitalized patients, Nutr. Clin. Pract. 2008; 23:373-382; Gibson, Principles of Nutritional Assessment. 2^{nd} ed. Oxford University Press, Inc; New York, NY, USA: 2005). Nutritional Risk Screening (NRS-2002) uses recent weight loss, decreased BMI and reduced dietary intake, combined with a subjective assessment of disease severity (based on increased nutrition requirements and/or metabolic stress), to generate a nutrition risk score (Anthony, Nutrition screening tools for hospitalized patients, Nutr. Clin. Pract. 2008; 23:373-382). The four item Short Nutrition Assessment Questionnaire (SNAQ) was developed to diagnose malnutrition in hospitalised patients and provides an indication for dietetic referrals as well as outlining a nutrition treatment plan (Anthony, Nutrition screening tools for hospitalized patients, Nutr. Clin. Pract. 2008; 23:373-382; Kruizenga et al., Development and validation of a hospital screening tool for malnutriton: the short nutritional assessment questionnaire (SNAQ) Clin. Nutr. 2005; 24:75-82). It has been validated for hospital inpatient and outpatient use, as well as residential patients and does not require calculation of BMI (Kruizenga et al. The SNAQ(RC), an easy traffic light system as a first step in the recognition of undernutrition in residential care. J. Nutr. Health Aging. 2010; 14:83-89; Neelemaat et al., Screening malnutrition in hospital outpatients. Can the SNAQ malnutrition screening tool also be applied to this population? Clin. Nutr. 2008; 27:439-446). Subjective Global Assessment (SGA) is one of the most commonly used nutrition assessment tools, and assesses nutrition status via completion of a questionnaire which includes data on weight change, dietary intake change, gastrointestinal symptoms, changes in functional capacity in relation to malnutrition as well as assessment of fat and muscle stores and the presence of oedema and ascites (Detsky et al., What is subjective global assessment of nutritional status? J Parenter Enteral Nutr. 1987; 11:8-13).

Hospital malnutrition is a condition experienced by a subject admitted to a hospital. The state of malnutrition may be pre-existing or develop during the subject's hospital stay. The present invention provides methods for treating or preventing hospital malnutrition in a subject comprising administering a therapeutically effective amount of LEPR/GDF8/ActA combinations (e.g., H4H18457P2/H4H1657N2/H4H10446P2) to the subject.

Malnutrition is also a common condition suffered by subjects with cystic fibrosis. A variety of complex factors, both related and unrelated, may give rise to energy imbalance in patients with cystic fibrosis. The net effect on growth potential varies considerably from patient to patient according to differences in disease expression and with progression of the disease. The present invention provides methods for treating or preventing malnutrition in a subject suffering from cystic fibrosis comprising administering a therapeutically effective amount of LEPR/GDF8/ActA combinations (e.g., H4H18457P2/H4H1657N2/H4H10446P2) to the subject.

Failure to thrive in childhood is a state of undernutrition due to inadequate caloric intake, inadequate caloric absorption, or excessive caloric expenditure. In newborns, failure to thrive may be associated with common underlying diseases such as short bowel following necrotizing enterocolitis, volvulus and intestinal resections, congenital resorption defects and structural defects of the small intestine and insufficient food intake. In infants (2-8 months of age), failure to thrive may be associated with common underlying diseases such as insufficient food intake, neglect, intestinal allergy to cow's milk protein, esophagitis with gastroesophogeal reflux, cystic fibrosis, eating disorders and/or increased energy requirements in case of underlying cardiac, neurological, oncological or renal disease, celiac disease, chronic diarrhea in case of immune-system defects, autoimmune enteropathy, postenteritis syndrome and malabsorption syndromes and munchausen syndrome by proxy. In small children (9-36 months), failure to thrive may be associated with common underlying diseases such as insufficient food intake, neglect, celiac disease, cystic fibrosis, eating disorders and/or increased energy requirements in case of underlying cardiac, neurological, oncological or renal disease, chronic diarrhea in case of immune-system defects and munchausen syndrome by proxy. In children (3-16 years), failure to thrive may be associated with common underlying diseases such as insufficient food intake, neglect, psychiatric disorders such as anorexia nervosa, chronic inflammatory intestinal disorders, celiac disease, cystic fibrosis, eating disorders and/or increased energy requirements in case of underlying cardiac, neurological, oncological or renal disease, chronic diarrhea in case of immune-system defects and lambliasis and other chronic intestinal infections. The present invention provides methods for treating or preventing childhood failure to thrive in a subject (e.g., in a newborn, infant, small child or child), for example, which is characterized by any one or more of the diseases or conditions discussed herein, comprising administering a therapeutically effective amount of LEPR/GDF8/ActA combinations (e.g., H4H18457P2/H4H1657N2/H4H10446P2) to the subject.

Insufficient food intake may be associated with the following symptoms: lack of appetite, chronic vomiting, swallowing and chewing disorders, esophageal dysmotility and shortness of breath e.g., associated with heart and lung disorders. The present invention provides methods for treating or preventing insufficient food intake, e.g., which is associated with the symptoms discussed herein, in a subject comprising administering a therapeutically effective amount of LEPR/GDF8/ActA combinations (e.g., H4H18457P2/H4H1657N2/H4H10446P2) to the subject.

Eating disorders characterized by inadequate caloric intake include anorexia and/or bulimia. The present invention provides methods for treating or preventing anorexia and/or bulimia in a subject comprising administering a therapeutically effective amount of LEPR/GDF8/ActA combinations (e.g., H4H18457P2/H4H1657N2/H4H10446P2) to the subject. In an embodiment of the invention, anorexia is anorexia nervosa, anorexia of aging, anorexia in a patient receiving hemodialysis.

Cachexia is a complex metabolic syndrome often associated with underlying illness and characterized by loss of muscle with or without loss of fat mass. The prominent clinical feature of cachexia is weight loss in adults (corrected for fluid retention) or growth failure in children (excluding endocrine disorders). Anorexia, inflammation, insulin resistance and increased muscle protein breakdown are symptoms frequently associated with cachexia. The present invention provides methods for treating or preventing cachexia (or any symptoms of cachexia), e.g., at any stage set forth herein (e.g., refractory cachexia) and/or as defined by any of the criteria set forth herein or in the art, in a subject comprising administering a therapeutically effective amount of LEPR/GDF8/ActA combinations (e.g., H4H18457P2/H4H1657N2/H4H10446P2) to the subject.

Cachexia secondary to other diseases, conditions or treatments includes cachexia secondary to anorexia or other psychiatric eating disorders, pulmonary disease (e.g., chronic obstructive pulmonary disorder (COPD)), chronic kidney disease, infectious disease (e.g., HIV-infection or acquired immune deficiency syndrome (AIDS)), congestive heart failure, radiation treatment, cancer (e.g., hepatocellular carcinoma, melanoma and/or breast cancer), chronic heart failure, autoimmune disorders (e.g., inflammatory bowel disease, lupus erythematosus, multiple sclerosis, rheumatoid arthritis, Crohn's disease or psoriasis), cystic fibrosis, cardiovascular diseases, elevated blood pressure, depression and/or neurodegenerative disorders. The present invention provides methods for treating or preventing cachexia which is secondary to any disease or condition, e.g., any of the disease or conditions set forth herein (e.g., cancer), in a subject comprising administering a therapeutically effective amount of LEPR/GDF8/ActA combinations (e.g., H4H18457P2/H4H1657N2/H4H10446P2) to the subject.

The international consensus statement on the definition and classification of cancer cachexia, published in May 2011 in Lancet Oncology, established these criteria for diagnosing cachexia in patients with cancer:
(i) Weight loss greater than 5 percent over the past 6 months; or
(ii) BMI less than 20 and any degree of weight loss greater than 2 percent; or
(iii) Appendicular skeletal muscle index consistent with sarcopenia (another wasting syndrome) and weight loss of more than 2 percent. Fearon et al., Lancet Oncol. 2011 May; 12(5):489-95.

The stages of cancer cachexia agreed upon by the panel are:
precachexia: weight loss of less than 5 percent, along with other symptoms such as impaired glucose tolerance or anorexia;
cachexia: weight loss greater than 5 percent or other symptoms and conditions consistent with the diagnostic criteria for cachexia; and
refractory cachexia: patients experiencing cachexia who are no longer responsive to cancer treatment, have a low performance score, and have a life expectancy of less than 3 months.

Muscle atrophy or wasting occurs in muscles with denervation or inactivity, but is also a systemic response to fasting and various diseases and conditions. Muscle atrophy may be in the form of myopenia, a decline in muscle mass, and/or dynapenia, a decline in muscle strength. These diseases and conditions include sepsis, AIDS, renal and cardiac failure, excessive glucocorticoids (e.g., Cushing syndrome) and trauma, and muscle atrophy also occurs in 80% of patients with cancer. In addition, muscle atrophy or wasting may be caused by or associated with disuse, immobilization, bed rest, injury (e.g., hip fracture), neurodegenerative diseases associated with motoneuron loss, medical treatment or surgical intervention (e.g., hip replacement, knee replacement, etc.) or by necessity of mechanical ventilation. The present invention provides methods for treating or preventing muscle atrophy or wasting (e.g., which is secondary to a disease or condition such as, for example, AIDS or cancer), in a subject comprising administering a therapeutically effective amount of LEPR/GDF8/ActA combinations (e.g., H4H18457P2/H4H1657N2/H4H10446P2) to the subject. In an embodiment of the invention, the muscle atrophy is a sarcopenia such as age-related sarcopenia or sarcopenia in a subject receiving hemodialysis and/or suffering from chronic kidney disease cachexia. Age-related sarcopenia is the degenerative loss of skeletal muscle mass (e.g., about 0.5-1% loss per year after the age of 50), quality, and strength associated with aging. Thus, the present invention includes method for treating or preventing muscle atrophy due to age-related sarcopenia.

Muscle injury may be caused by strain from overexertion or a sudden twist, e.g., a strain or a tear. A muscle tear might cause swelling, pain, and severe bleeding, which can lead to a blood clot. Serious tears may require surgery. The muscle stimulating properties of the LEPR/GDF8/ActA combinations make them useful for the treatment or prevention of muscle injury. The present invention provides methods for treating or preventing muscle injury, in a subject, comprising administering a therapeutically effective amount of LEPR/GDF8/ActA combinations (e.g., H4H18457P2/H4H1657N2/H4H10446P2) to the subject.

Other conditions that may be treated or prevented using the LEPR/GDF8/ActA combinations of the present invention include amyotrophic lateral sclerosis, arthritis, autoimmune disorders, benign and malignant pheochromocytoma, breast cancer, cancer, cardiovascular diseases, chronic heart failure, chronic obstructive pulmonary disease, depression, diabetes, elevated blood pressure, glucocorticoid-induced myopathy, hepatocellular carcinoma, inflammatory bowel disease, keloids and hypertrophic scars, lupus erythematosus, melanoma, metabolic syndromes, multiple sclerosis, muscular dystrophy (e.g., Myotonic, Duchenne, Becker, Limb-girdle, Facioscapulohumeral (FSHD, also known as Landouzy-Dejerine disease), Congenital, Oculopharyngeal, Distal, Emery-Dreifuss, etc.), neurodegenerative disorders, organ atrophy, osteoarthritis, osteopenia, osteoporosis, Parkinson's disease, preeclampsia, psoriasis, pulmonary artery hypertension, sarcopenia, sepsis and uterine fibroids/leiomyomata.

LEPR/GDF8/ActA combinations (e.g., H4H18457P2/H4H1657N2/H4H10446P2) of the present invention are effective for increasing lean muscle mass and muscle strength and, thus, are effective for enhancing athletic performance. Thus, the present invention provides methods for enhancing athletic performance in a subject in need thereof comprising administering a therapeutically effective amount of LEPR/GDF8/ActA combinations (e.g., H4H18457P2/H4H1657N2/H4H10446P2) to the subject. Athletic performance includes walking speed, running speed, ability to sit and stand per unit time (e.g., over 30 seconds), walking distance per unit time (e.g., over 6 minutes), bicep curl weight, chest press weight, stair climb speed (e.g., time to climb 4 steps) and/or hand grip strength (e.g., as measured using manual dynamometry). In an embodiment of the invention, the subject is undergoing stroke rehabilitation (e.g., rehabilitation for stroke hemiparesis) or physical therapy. Thus, the LEPR/GDF8/ActA combinations of the present invention can be used as an adjunct to any therapeutic procedure wherein an increase in athletic performance would be desirable, e.g., physical therapy, for example, stroke rehabilitation or for recovery from surgery (e.g., knee surgery to repair tendon or ligament damage or knee replacement) or a physical injury.

Subjects (e.g., humans) administered leptin receptor antagonists (e.g., anti-LEPR antibodies) may experience increased liver triglyceride levels or serum triglyceride levels. One method for mitigating this increase is to administer, in association with the LEPR antagonist, an Activin A antagonist (e.g., anti-ActA antibody or antigen-binding fragment thereof) and a GDF8 antagonist (e.g., anti-GDF8 antibody or antigen-binding fragment thereof). For example, such a method may comprise administering a therapeutically effective amount of a LEPR/GDF8/ActA combination (e.g., H4H18457P2/H4H1657N2/H4H10446P2).

A “subject” is a mammal such as, for example, a human, dog, cat, horse, cow, mouse, rat, monkey (e.g., cynomolgous monkey, e.g., Macaca fascicularis or Macaca mulatta) or rabbit.

An effective or therapeutically effective dose of LEPR/GDF8/ActA combinations (e.g., H4H18457P2/H4H1657N2/H4H10446P2) of the present invention, is from about 0.1 to about 200 mg/kg (of all three antibodies or antigen-binding fragments), e.g., for treatment or prevention of any of the diseases or conditions discussed herein (e.g., cachexia).

In particular embodiments, the LEPR/GDF8/ActA combinations of the present invention (e.g., H4H18457P2/H4H1657N2/H4H10446P2) may be used alone or in association with any other, further therapeutic agents and/or therapeutic procedures, e.g., which are useful for increasing food intake, adiposity, body weight, lean mass (e.g., muscle mass) and/or strength in a subject and/or for treating or preventing any of the diseases or conditions discussed herein, e.g., cachexia.

In an embodiment of the invention, the therapeutic procedure is nasogastric tube feeding.

In an embodiment of the invention, the further therapeutic agent is any one or more of an appetite stimulant, a cannabinoid, an angiotensin-converting enzyme (ACE) inhibitor, an angiotensin receptor blocker, a smooth muscle relaxant, a nitrate, a diuretic, iron, a bronchodilator, an anticholinergic, a corticosteroid, an antibiotic, a nonsteroidal anti-inflammatory drug (NSAID), an immunosuppressant, an HMG-CoA reductase inhibitor, an anti-depressant, an anti-cancer therapy or a topical agent.

In an embodiment of the invention, the further therapeutic agent is any one or more of 5-fluorouracil (5-FU), 6-mercaptopurine, a combination of atorvastatin and amlodipine, a combination of lovastatin and niacin, a combination such as simvastatin and ezetimibe, atropine, adalimumab, albuterol, alefacept, alemtuzumab, amitriptyline, anamorelin, arformoterol, aspirin, aspirin, atorvastatin, atropine, azathioprine, azithromycin, benazepril, betamethasone, budesonide, bumetanide, buphenine, bupropion, captopril, carboplatin, celecoxib, certolizumab pegol, chlorothiazide, chlorthalidone, ciclosporin, citalopram, clenbuterol, coal tar, coconut oil, corticosteroids, cyproheptadine, cyclophosphamide, dabrafenib, daclizumab, desoximetasone, desvenlafaxine, dexamethasone, diclofenac, dicyclomine, diflunisal, dimebolin, dimethyl fumarate, dimethyl fumarate, dithranol, docetaxel, dopexamine, doxorubicin, dronabinol, duloxetine, efalizumab, enalapril, enobosarm, epinephrine, epirubicin, erythromycin, escitalopram, etanercept, ethacrynate, etodolac, fentoterol, fingolimod, flunisolide, fluocinonide, fluocortolone, fluoxetine, fluvastatin, formoterol, fosinopril, furosemide, glatiramer acetate, golimumab, hydralazine, hydrochlorothiazide, hydrocortisone, hydrocortisone-17-butyrate, hydrocortisone-17-valerate, hydroxycarbamide, hyoscyamine, hyoscyamine, ibuprofen, indapamide, indomethacin, infliximab, interferon beta-1a, interferon beta-1b, interleukin-2, ipilimumab, isoetarine, isoprenaline, isoproterenol, isosorbide dinitrate, JX-594, ketoprofen, levosalbutamol, lisinopril, lovastatin, megestrol, megestrol acetate, mepenzolate, mesalazine, methotrexate, methotrexate, methyclothiazide, metolazone, mevastatin, mitoxantrone, moexipril, nabilone, naproxen, natalizumab, nivolumab, nortriptyline, ocrelizumab, ofatumumab, oral nutritional supplements (e.g., Protibis cookies or dairy-based supplements), orciprenaline, oxaprozin, paclitaxel, para-aminobenzoic acid, parenteral iron, paroxetine, pembrolizumab, perindopril, phenobarbital, phenobarbital, phenobarbital and scopolamine, pirbuterol, piroxicam, pitavastatin, pravastatin, prednisolone, prednisone, prednisone, procaterol, proline-rich peptide (PRP)-1, psoralen, quinapril, ramapril, ritodrine, rituximab, rosuvastatin, salbutamol, salsalate, scopolamine, sertraline, simvastatin, sorafenib, sulindac, terbutaline, teriflunomide, theophylline, tiotropium, tolmetin, torsemide, trametinib, trandolapril, trazodone, triamcinolone acetonide, triamcinolone alcohol, vemurafenib, venlafacine, venlafaxine, vitamin D3 or a vitamin D analogue.

The present invention also encompasses embodiments wherein a LEPR/GDF8/ActA combination is not in association with a further therapeutic agent and/or procedure.

### EXAMPLES

These examples are intended to exemplify the present invention are not a limitation thereof. Compositions e.g., LEPR/GDF8/ActA combinations, and methods set forth in the Examples form part of the present invention.

### Example 1: In Vivo Efficacy Testing of LEPR Antagonist Antibodies (H4H17322P2, H4H18457P2 and H4H18464P2) in Humanized LEPR Mice

The effects of three specific antagonist anti-LEPR antibodies of the invention, H4H17322P2, H4H18457P2 and H4H18464P2, on food intake, body weight and adiposity were determined in singly-housed genetically engineered LEPR^{Hu/Hu} mice that express a leptin receptor which is composed of the human LEPR ectodomain sequence in place of the murine LEPR ectodomain sequence.

Baseline daily food intake was measured between 5 days and 1 day prior to treatment (days −5 and −1). Four days prior to treatment and 6 days post treatment (days −4 and 6) body composition, including adiposity, was quantified by μCT. At day 0, thirty-two 12- to 13-week old male LEPR^{Hu/Hu} mice were randomized to four groups of 8 mice based on body weight from 1-day pretreatment (day −1). At day 0, each group received via subcutaneous injection either a single dose of isotype control antibody at 30 mg/kg, H4H17322P2 at 30 mg/kg, H4H18457P2 at 30 mg/kg, or H4H18464P2 at 30 mg/kg. The isotype control antibody does not bind any known mouse protein. Body weight was measured for the duration of the study for each animal (Table 1A). The percent change in body weight from day 0 was calculated for each animal at each time point. Table 1B summarizes the average fat mass and lean mass for animals in each antibody treatment group quantified by μCT 6 days prior to and 6 days following antibody treatment. All results are expressed as mean±SEM. Moreover, plasma leptin, both pre-dose and at day 6 were quantitated (Table 1C).

Mice treated with the anti-LEPR antagonist antibodies demonstrated increases in percent change in food intake (data not shown) and change in body weight (Table 1A). These increases were not observed with the isotype control antibody treatment. Mice treated with H4H17322P2 at 30 mg/kg exhibited significant increases in food intake starting at one day after treatment (day 1) and at the subsequent time points compared to mice injected with isotype control antibody. Mice treated with H4H18457P2 at 30 mg/kg exhibited a significant increase in food intake starting at day 2 and at the subsequent time points compared to mice injected with isotype control antibody. Mice treated with H4H18464P2 at 30 mg/kg exhibited a significant increase in food intake starting at day 2 and at the subsequent time points, but not day 4 compared to mice injected with isotype control antibody. Mice treated with H4H17322P2 at 30 mg/kg exhibited a significant increase in percent body weight change starting four days after treatment (day 4) and at the subsequent time points compared to mice injected with isotype control antibody. Mice treated with H4H18457P2 at 30 mg/kg exhibited a significant increase in percent body weight change starting at day 3 and at the subsequent time points compared to mice injected with isotype control antibody. Mice treated with H4H18464P2 at 30 mg/kg exhibited a significant increase in percent body weight change starting at day 4 and at subsequent time points compared to mice injected with isotype control antibody. As depicted in Table 1B, there were no differences in fat mass between the groups prior to treatment (day −4). Mice treated with H4H17322P2, H4H18457P2 and H4H18464P2 antibody at 30 mg/kg demonstrated a significant increase in fat mass and leptin levels at 6 days after treatment (day 6) as compared to isotype control antibody (Table 1C). In conclusion, treatment with LEPR antagonist antibody, but not an isotype control antibody, increased food intake, body weight, adiposity and leptin levels in mice.

TABLE 1A
Body Weight (g Difference from Baseline)

| | REGN1945 30 mg/kg | | | H4H18457 30 mg/kg | | | H4H18464 30 mg/kg | | | H4H17322 30 mg/kg | | |
|---|---|---|---|---|---|---|---|---|---|---|---|---|
| | Mean | SEM | N | Mean | SEM | N | Mean | SEM | N | Mean | SEM | N |
| Day 0 | 0.000 | 0.000 | 12 | 0.000 | 0.000 | 12 | 0.000 | 0.000 | 12 | 0.000 | 0.000 | 12 |
| Day 1 | 0.088 | 0.137 | 12 | 0.597 | 0.149 | 12 | 0.300 | 0.153 | 12 | 0.331 | 0.142 | 12 |
| Day 4 | −0.052 | 0.322 | 12 | 2.505 | 0.158 | 12 | 2.215 | 0.212 | 12 | 1.169 | 0.181 | 12 |
| Day 5 | 0.405 | 0.144 | 12 | 2.679 | 0.204 | 12 | 2.433 | 0.239 | 12 | 1.178 | 0.194 | 12 |
| Day 6 | 0.093 | 0.171 | 12 | 2.563 | 0.282 | 12 | 2.062 | 0.229 | 12 | 1.021 | 0.180 | 12 |
| Day 8 | 0.563 | 0.128 | 12 | 3.243 | 0.323 | 12 | 2.959 | 0.360 | 12 | 1.710 | 0.364 | 12 |
| Day 11 | 0.289 | 0.283 | 12 | 3.604 | 0.367 | 12 | 2.927 | 0.434 | 12 | 2.165 | 0.299 | 12 |
| Day 13 | 0.356 | 0.233 | 12 | 3.091 | 0.413 | 12 | 2.116 | 0.317 | 12 | 1.678 | 0.336 | 12 |

REGN1945: Anti-Fel d1 (IgG4)
SEM: standard error of the mean

TABLE 1B
Body Composition

| | REGN1945 30 mg/kg | | | H4H18457 30 mg/kg | | | H4H18464 30 mg/kg | | | H4H17322 30 mg/kg | | |
|---|---|---|---|---|---|---|---|---|---|---|---|---|
| | Mean | SEM | N | Mean | SEM | N | Mean | SEM | N | Mean | SEM | N |
| Fat Mass (g Difference | 0.033 | 0.141 | 12 | 1.563 | 0.168 | 12 | 1.396 | 0.217 | 12 | 0.767 | 0.123 | 12 |
| from Baseline) | | | | | | | | | | | | |
| Lean Mass (g Difference | 0.576 | 0.485 | 12 | 0.718 | 0.153 | 12 | 0.724 | 0.297 | 12 | 0.624 | 0.331 | 12 |
| from Baseline) | | | | | | | | | | | | |

TABLE 1C
Plasma Leptin (pg/mL)

| | REGN1945 30 mg/kg | | | H4H18457 30 mg/kg | | | H4H18464 30 mg/kg | | | H4H17322 30 mg/kg | | |
|---|---|---|---|---|---|---|---|---|---|---|---|---|
| | Mean | SEM | N | Mean | SEM | N | Mean | SEM | N | Mean | SEM | N |
| Predose | 690.590 | 101.890 | 12 | 1202.452 | 293.789 | 12 | 1068.469 | 224.895 | 12 | 952.510 | 190.816 | 12 |
| Day 6 | 983.476 | 136.172 | 12 | 5956.415 | 977.674 | 12 | 4467.408 | 651.147 | 12 | 3630.862 | 442.252 | 12 |

### Example 2: Combination Treatment of Anti-GDF8 mAb (REGN1033), Anti-Activin a mAb (REGN2477), and LEPR Antagonist mAb (H4H184572P2) in 20-24 Week Old Male Mice

The effects of the specific antagonist anti-LEPR antibody, H4H18457P2, in combination with the anti-MSTN (also referred to as anti-GDF8) and anti-INHBA (also referred to as anti-Activin A) blocking antibodies, H4H1657N2 (REGN1033) and H4H10446P2 (REGN2477), respectively, of the invention, on food intake, body weight, body composition, individual tissue weights, and ex vivo muscle force generation were determined in singly-housed genetically engineered 20 to 24 week old male LEPR^{Hu/Hu} mice, that express a leptin receptor which is composed of the human LEPR ectodomain sequence in place of the murine LEPR ectodomain sequence.

Baseline daily food intake was measured between days −8 and 0. On day −5 or day −1, baseline whole body lean and fat mass was quantified by NMR (nuclear magnetic resonance). On day 0, mice were stratified to four groups of 11 to 12 mice based on body composition from day −5 and body weight from day 0. Starting on day 0, each group received the respective antibody treatment dose via subcutaneous injection. REGN1033, REGN2477, and the respective IgG4^{P} mAbs were dosed twice a week at 10 mg/kg; and H4H18457P2 and its respective IgG4^{P} mAb were dosed once a week at 30 mg/kg; thus, any dose of test antibody given to a test mouse was given in parallel with a corresponding dose of control antibody in a control mouse. The isotype control (IgG4^{P}) (^{P}=denotes a S228P mutation, Eu numbering) antibody did not bind any known mouse protein. The isotype control antibody was REGN1945.

### Treatment Groups

$$\\ \begin{matrix}IgG {4}^{{}_{} P} Control ( \begin{matrix}10 mg / kg + 10 mg / kg , 2 x / week ; \\ 30 mg / kg , 1 x / week\end{matrix} ) , N = 11 & a )\end{matrix}$$

$$\\ \begin{matrix}REGN 1033 + REGN 2477 ( 10 mg / kg + 10 mg / kg , 2 x / week ) , N = 11 & b )\end{matrix}$$

$$\\ \begin{matrix}H 4 H 18457 P 2 ( 30 mg / kg , 1 x / week ) , N = 11 & c )\end{matrix}$$

$$\\ \begin{matrix}REGN 1033 + REGN 2477 + 4 H 18457 P 2 ( \begin{matrix}10 mg / kg + 10 mg / kg , 2 x / week ; \\ 30 mg / kg , 1 x / week )\end{matrix} ) . & d )\end{matrix}$$

Food intake (Table 2A) and body weight (Table 2B) were measured for the duration of the study for each animal. Body composition (Tables 2C and 4A-4D) was quantified on day 6 or 7 and day 13 or 14. On day 22, 23 or 24, animals were euthanized and ex vivo force measurements on isolated tibialis anterior muscled were performed (Tables 3A and 3B). Individual organ and skeletal muscle weights were also quantified (Tables 5-7).

Mice treated with either H4H18457P2 alone or in combination with the REGN1033 and REGN2477 exhibited significant increases in cumulative food intake starting at seven days after treatment (day 7) and at the subsequent time points compared to mice injected with isotype control antibody (Table 2A). Mice treated with REGN1033 and REGN2477 showed similar cumulative food intake compared to mice injected with isotype control antibody (Table 2A). Mice treated with H4H18457P2 alone or in combination with REGN1033 and REGN2477, demonstrated increases in body weight beginning 7 days after dosing (day 7) and at the subsequent time points compared to mice treated with isotype control antibody and when compared to mice treated with REGN1033 and REGN2477 (Table 2B). Mice treated with REGN1033 and REGN2477 showed no significant changes in body weight compared to mice treated with isotype control antibody (Table 2B). A significant increase in lean mass from baseline (day −1) was exhibited by mice in each treatment group on days 6, 13 and 20 when compared to mice injected with isotype control (Table 4C). Mice treated with H4H18457P2, REGN1033 and REGN2477 exhibited increased lean mass gain from baseline on days 6, 13 and 20 compared to mice treated with REGN1033 and REGN2477 (Table 4C). Mice treated with REGN1033 and REGN2477 showed a significant decrease in fat mass change from baseline when compared to mice administered isotype control antibody. Mice treated with either H4H18457P2 alone or in combination with the REGN1033 and REGN2477 showed increased fat mass change from baseline at days 6, 13 and 20 when compared to mice administered isotype control antibody and when compared to mice treated with REGN1033 and REGN2477 (Table 4A). Mice treated with H4H18457P2 in combination with REGN1033 and REGN2477 showed a significant reduction in fat mass gain from baseline at days 6, 13 and 20 when compared to mice treated with REGN1033 and REGN2477 (Table 4A). In comparison to mice administered isotype control antibody, mice treated with REGN1033 and REGN2477, as well as mice treated with H4H18457P2, REGN1033 and REN2477 exhibited increased twitch force and peak tetanic force (Table 3A) of isolated tibialis anterior skeletal muscle. Accordingly, mice treated with REGN1033 and REGN2477 and mice treated with H4H18457P2, REGN1033 and REGN3477 exhibited increased skeletal muscle (quadriceps, tibialis anterior and gastrocnemius) weights (Table 5) when compared to mice administered isotype control antibody. Mice treated with H4H18457P2 alone and in combination with REGN1033 and REGN2477 showed increased inguinal, gonadal and brown adipose tissue weights (Table 5) compared to mice administered isotype control antibody. Brown adipose tissue weights (Table 5) were significantly increased in mice treated with H4H18457P2 in combination with REGN1033 and REGN2477 than mice treated with REGN1033 and REGN2477. No significant changes in heart and liver weights (Table 5) were detected amongst the treatment groups when compared to isotype control administration. In summary, these data demonstrate that the LEPR antagonist, H4H18457P2, increases food intake, body weight and increases fat mass in mice. In addition, treatment with the combination of LEPR antagonist, H4H18457P2, with anti-MSTN (REGN1033) and anti-ActA (REGN2477) blocking antibodies leads to increased food intake, body weight, lean mass, fat mass, adipose tissue weight, skeletal muscle weight, and skeletal muscle strength. When compared to REGN1033 and REGN2477 treatment, the combined treatment with H4H18457P2, REGN1033 and REGN2477 leads to additional lean mass increases from baseline and smaller increases in fat mass from baseline.

TABLE 2A
Food Intake (g)

| | | | | | | | | | | LEPR antagonist | | |
|---|---|---|---|---|---|---|---|---|---|---|---|---|
| | | | | | | | LEPR antagonist | | | mAb + αGDF8 + | | |
| | Control mAb | | | αGDF8 + αActA | | | mAb | | | αActA | | |
| | Mean | SEM | N | Mean | SEM | N | Mean | SEM | N | Mean | SEM | N |
| Day 0 | 0.000 | 0.000 | 11 | 0.000 | 0.000 | 11 | 0.000 | 0.000 | 11 | 0.000 | 0.000 | 12 |
| Day 1 | 3.858 | 0.274 | 11 | 3.987 | 0.158 | 11 | 4.554 | 0.380 | 11 | 4.264 | 0.204 | 12 |
| Day 2 | 7.958 | 0.444 | 11 | 8.154 | 0.294 | 11 | 9.784 | 0.679 | 11 | 9.224 | 0.371 | 12 |
| Day 3 | 12.148 | 0.573 | 11 | 12.330 | 0.422 | 11 | 15.246 | 0.919 | 11 | 14.618 | 0.502 | 12 |
| Day 7 | 28.465 | 1.132 | 11 | 28.667 | 0.887 | 11 | 36.752 | 2.059 | 11 | 35.921 | 1.052 | 12 |
| Day 8 | 32.434 | 1.299 | 11 | 32.585 | 1.039 | 11 | 42.095 | 2.394 | 11 | 41.094 | 1.109 | 12 |
| Day 9 | 36.393 | 1.427 | 11 | 36.773 | 1.131 | 11 | 47.590 | 2.737 | 11 | 46.163 | 1.143 | 12 |
| Day 10 | 40.386 | 1.566 | 11 | 40.853 | 1.221 | 11 | 52.779 | 2.874 | 11 | 51.400 | 1.263 | 12 |
| Day 14 | 56.776 | 1.935 | 11 | 57.395 | 1.640 | 11 | 73.535 | 3.966 | 11 | 71.676 | 1.586 | 12 |
| Day 15 | 60.708 | 2.059 | 11 | 61.353 | 1.802 | 11 | 78.424 | 4.353 | 11 | 76.398 | 1.604 | 12 |
| Day 16 | 64.485 | 2.092 | 11 | 65.544 | 1.868 | 11 | 83.396 | 4.662 | 11 | 81.271 | 1.646 | 12 |
| Day 17 | 68.394 | 2.182 | 11 | 69.716 | 1.951 | 11 | 88.506 | 4.993 | 11 | 86.352 | 1.730 | 12 |
| Day 20 | 80.272 | 2.547 | 11 | 82.074 | 2.267 | 11 | 103.698 | 5.970 | 11 | 101.008 | 1.918 | 12 |
| Day 21 | 83.961 | 2.518 | 11 | 85.904 | 2.326 | 11 | 108.243 | 6.360 | 11 | 105.226 | 2.016 | 12 |

TABLE 2B
Body Weight (g Difference from Baseline)

| | | | | | | | | | | LEPR antagonist | | |
|---|---|---|---|---|---|---|---|---|---|---|---|---|
| | | | | | | | LEPR antagonist | | | mAb + αGDF8 + | | |
| | Control mAb (N = 11) | | | αGDF8 + αActA | | | mAb | | | αActA | | |
| | Mean | SEM | N | Mean | SEM | N | Mean | SEM | N | Mean | SEM | N |
| Day 0 | 0.000 | 0.000 | 11 | 0.000 | 0.000 | 11 | 0.000 | 0.000 | 11 | 0.000 | 0.000 | 12 |
| Day 1 | −0.030 | 0.092 | 11 | 0.052 | 0.064 | 11 | 0.614 | 0.172 | 11 | 0.405 | 0.158 | 12 |
| Day 2 | 0.324 | 0.140 | 11 | 0.504 | 0.104 | 11 | 1.355 | 0.218 | 11 | 1.431 | 0.210 | 12 |
| Day 3 | 0.222 | 0.162 | 11 | 0.644 | 0.107 | 11 | 1.791 | 0.224 | 11 | 2.273 | 0.187 | 12 |
| Day 7 | −0.008 | 0.133 | 11 | 1.314 | 0.152 | 11 | 3.192 | 0.327 | 11 | 4.383 | 0.288 | 12 |
| Day 8 | 0.089 | 0.134 | 11 | 1.585 | 0.177 | 11 | 3.559 | 0.288 | 11 | 4.772 | 0.348 | 12 |
| Day 9 | 0.139 | 0.189 | 11 | 1.908 | 0.151 | 11 | 4.088 | 0.380 | 11 | 5.251 | 0.361 | 12 |
| Day 10 | −0.106 | 0.141 | 11 | 1.849 | 0.171 | 11 | 4.244 | 0.374 | 11 | 5.489 | 0.296 | 12 |
| Day 14 | 0.217 | 0.248 | 11 | 2.229 | 0.217 | 11 | 4.931 | 0.417 | 11 | 6.666 | 0.299 | 12 |
| Day 15 | 0.171 | 0.265 | 11 | 2.215 | 0.188 | 11 | 5.321 | 0.502 | 11 | 6.964 | 0.332 | 12 |
| Day 16 | 0.281 | 0.262 | 11 | 2.576 | 0.191 | 11 | 5.564 | 0.504 | 11 | 7.357 | 0.326 | 12 |
| Day 17 | 0.050 | 0.265 | 11 | 2.395 | 0.178 | 11 | 5.535 | 0.492 | 11 | 7.386 | 0.302 | 12 |
| Day 20 | 0.195 | 0.345 | 11 | 2.626 | 0.207 | 11 | 6.429 | 0.449 | 11 | 8.196 | 0.378 | 12 |
| Day 21 | −0.347 | 0.285 | 11 | 2.172 | 0.193 | 11 | 6.024 | 0.502 | 11 | 7.624 | 0.363 | 12 |

TABLE 2C
Change in Body Composition from Baseline

| | Lean Mass Change (g) | | | Fat Mass Change (g) | | |
|---|---|---|---|---|---|---|
| | Mean | SEM | N | Mean | SEM | N |
| Control mAb | −0.467 | 0.210 | 11 | 0.286 | 0.118 | 11 |
| αGDF8 + αActA | 3.432 | 0.117 | 11 | −1.118 | 0.205 | 11 |
| LEPR antagonist mAb | 0.532 | 0.195 | 11 | 4.897 | 0.551 | 11 |
| LEPR antagonist mAb + | 3.992 | 0.288 | 12 | 3.400 | 0.298 | 12 |
| αGDF8 + αActA | | | | | | |

TABLE 3A
Twitch Force, Peak Tetanic Force, and Specific Force (mN)

| | | | | | | | LEPR antagonist | | | LEPR antagonist | | |
|---|---|---|---|---|---|---|---|---|---|---|---|---|
| | Control mAb | | | αGDF8 + αActA | | | mAb | | | mAb + αGDF8 + αActA | | |
| | Mean | SEM | N | Mean | SEM | N | Mean | SEM | N | Mean | SEM | N |
| Twitch Force (mN) | 352.300 | 16.990 | 6 | 472.300 | 18.730 | 5 | 352.000 | 24.000 | 4 | 481.700 | 27.340 | 7 |
| Peak Tetanic Force (mN) | 997.300 | 47.510 | 6 | 1415.000 | 49.740 | 5 | 842.000 | 80.380 | 4 | 1282.000 | 59.600 | 7 |
| Specific Force (mN) | 19.770 | 0.317 | 6 | 20.500 | 0.150 | 5 | 15.770 | 1.661 | 4 | 19.200 | 1.092 | 7 |

TABLE 3B
Isometric Tetanic Force (mN)

| | | | | | | | | LEPR antagonist | | | LEPR antagonist | | |
|---|---|---|---|---|---|---|---|---|---|---|---|---|---|
| | | Control mAb | | | αGDF8 + αActA | | | mAb | | | mAb + αGDF8 + αActA | | |
| | | Mean | SEM | N | Mean | SEM | N | Mean | SEM | N | Mean | SEM | N |
| 40 | Hz | 564.712 | 48.438 | 6 | 771.410 | 37.949 | 5 | 582.244 | 28.587 | 4 | 788.626 | 58.501 | 7 |
| 60 | Hz | 882.634 | 43.772 | 6 | 1183.435 | 47.472 | 5 | 791.627 | 50.899 | 4 | 1142.551 | 51.231 | 7 |
| 80 | Hz | 976.495 | 47.602 | 6 | 1357.974 | 50.474 | 5 | 833.329 | 73.525 | 4 | 1249.999 | 60.957 | 7 |
| 100 | Hz | 995.798 | 47.934 | 6 | 1414.735 | 49.740 | 5 | 813.684 | 88.515 | 4 | 1282.284 | 59.602 | 7 |
| 125 | Hz | 979.120 | 47.815 | 6 | 1393.443 | 45.717 | 5 | 778.318 | 97.194 | 4 | 1244.875 | 57.801 | 7 |
| 150 | Hz | 926.029 | 45.454 | 6 | 1325.240 | 50.839 | 5 | 732.848 | 91.838 | 4 | 1174.292 | 59.950 | 7 |
| 200 | Hz | 847.518 | 42.148 | 6 | 1199.041 | 50.727 | 5 | 678.718 | 84.987 | 4 | 1070.346 | 60.009 | 7 |

TABLE 4A
Fat Mass (g)

| | | | | | | | | | | LEPR antagonist | | |
|---|---|---|---|---|---|---|---|---|---|---|---|---|
| | | | | | | | LEPR antagonist | | | mAb + αGDF8 + | | |
| | Control mAb | | | αGDF8 + αActA | | | mAb | | | αActA | | |
| | Mean | SEM | N | Mean | SEM | N | Mean | SEM | N | Mean | SEM | N |
| Week 0 | 5.227 | 0.477 | 11 | 5.779 | 0.558 | 11 | 5.626 | 0.461 | 11 | 5.425 | 0.415 | 12 |
| Week 1 | 5.149 | 0.503 | 11 | 5.199 | 0.528 | 11 | 7.476 | 0.626 | 11 | 6.583 | 0.510 | 12 |
| Week 2 | 5.395 | 0.544 | 11 | 4.858 | 0.484 | 11 | 9.389 | 0.759 | 11 | 8.008 | 0.401 | 12 |
| Week 3 | 5.514 | 0.491 | 11 | 4.661 | 0.458 | 11 | 10.524 | 0.800 | 11 | 8.825 | 0.411 | 12 |

TABLE 4B
Fat Mass (g Difference from Baseline)

| | | | | | | | | | | LEPR antagonist | | |
|---|---|---|---|---|---|---|---|---|---|---|---|---|
| | | | | | | | LEPR antagonist | | | mAb + αGDF8 + | | |
| | Control mAb | | | αGDF8 + αActA | | | mAb | | | αActA | | |
| | Mean | SEM | N | Mean | SEM | N | Mean | SEM | N | Mean | SEM | N |
| Week 0 | 0.000 | 0.000 | 11 | 0.000 | 0.000 | 11 | 0.000 | 0.000 | 11 | 0.000 | 0.000 | 12 |
| Week 1 | −0.078 | 0.096 | 11 | −0.580 | 0.086 | 11 | 1.850 | 0.234 | 11 | 1.158 | 0.257 | 12 |
| Week 2 | 0.167 | 0.123 | 11 | −0.921 | 0.160 | 11 | 3.763 | 0.410 | 11 | 2.583 | 0.304 | 12 |
| Week 3 | 0.286 | 0.118 | 11 | −1.118 | 0.205 | 11 | 4.897 | 0.551 | 11 | 3.400 | 0.298 | 12 |

TABLE 4C
Lean Mass (g)

| | | | | | | | | | | LEPR antagonist | | |
|---|---|---|---|---|---|---|---|---|---|---|---|---|
| | | | | | | | LEPR antagonist | | | mAb + αGDF8 + | | |
| | Control mAb | | | αGDF8 + αActA | | | mAb | | | αActA | | |
| | Mean | SEM | N | Mean | SEM | N | Mean | SEM | N | Mean | SEM | N |
| Week 0 | 23.507 | 0.476 | 11 | 23.075 | 0.493 | 11 | 23.213 | 0.637 | 11 | 23.420 | 0.486 | 12 |
| Week 1 | 23.390 | 0.517 | 11 | 24.701 | 0.554 | 11 | 23.805 | 0.613 | 11 | 25.693 | 0.492 | 12 |
| Week 2 | 23.307 | 0.547 | 11 | 25.968 | 0.590 | 11 | 23.681 | 0.547 | 11 | 26.865 | 0.565 | 12 |
| Week 3 | 23.040 | 0.495 | 11 | 26.507 | 0.507 | 11 | 23.745 | 0.539 | 11 | 27.412 | 0.545 | 12 |

TABLE 4D
Lean Mass (g Difference from Baseline)

| | | | | | | | | | | LEPR antagonist | | |
|---|---|---|---|---|---|---|---|---|---|---|---|---|
| | | | | | | | LEPR antagonist | | | mAb + αGDF8 + | | |
| | Control mAb | | | αGDF8 + αActA | | | mAb | | | αActA | | |
| | Mean | SEM | N | Mean | SEM | N | Mean | SEM | N | Mean | SEM | N |
| Week 0 | 0.000 | 0.000 | 11 | 0.000 | 0.000 | 11 | 0.000 | 0.000 | 11 | 0.000 | 0.000 | 12 |
| Week 1 | −0.117 | 0.123 | 11 | 1.625 | 0.129 | 11 | 0.592 | 0.110 | 11 | 2.273 | 0.116 | 12 |
| Week 2 | −0.200 | 0.148 | 11 | 2.893 | 0.195 | 11 | 0.468 | 0.140 | 11 | 3.445 | 0.186 | 12 |
| Week 3 | −0.467 | 0.210 | 11 | 3.432 | 0.117 | 11 | 0.532 | 0.195 | 11 | 3.992 | 0.288 | 12 |

TABLE 5
Organ Mass, Muscle Mass, Fat Mass (mg)

| | | | | | | | | | | LEPR antagonist | | |
|---|---|---|---|---|---|---|---|---|---|---|---|---|
| | | | | | | | LEPR antagonist | | | mAb + αGDF8 + | | |
| | Control mAb | | | αGDF8 + αActA | | | mAb | | | αActA | | |
| | Mean | SEM | N | Mean | SEM | N | Mean | SEM | N | Mean | SEM | N |
| Heart | 144.70 | 4.86 | 11 | 149.50 | 2.67 | 11 | 151.90 | 3.13 | 11 | 152.10 | 2.99 | 12 |
| Liver | 1449.00 | 71.30 | 10 | 1432.00 | 55.00 | 11 | 1695.00 | 105.20 | 11 | 1637.00 | 45.11 | 12 |
| Quad | 203.10 | 4.45 | 11 | 270.80 | 7.69 | 11 | 210.10 | 6.95 | 11 | 263.10 | 8.05 | 12 |
| TA Muscle | 51.07 | 1.59 | 11 | 65.33 | 2.01 | 11 | 51.35 | 1.80 | 11 | 67.41 | 1.89 | 12 |
| GA Muscle | 161.90 | 4.02 | 11 | 212.00 | 5.29 | 11 | 166.10 | 5.90 | 11 | 213.10 | 4.87 | 12 |
| Inguinal WAT | 510.50 | 60.33 | 11 | 430.30 | 56.39 | 11 | 992.20 | 84.22 | 11 | 893.60 | 67.38 | 12 |
| Gonadal WAT | 771.30 | 84.83 | 11 | 652.20 | 67.24 | 11 | 1592.00 | 80.00 | 11 | 1414.00 | 73.00 | 12 |
| BAT | 103.50 | 10.32 | 11 | 97.96 | 6.19 | 11 | 186.10 | 15.47 | 11 | 146.00 | 7.19 | 12 |

TA: tibialis anterior; GA: gastrocnemius; WAT: white adipose tissue; BAT: brown adipose issue.

TABLE 6
Organ Mass, Muscle Mass, Fat Mass (% of Starting Body Weight)

| | | | | | | | | | | LEPR antagonist | | |
|---|---|---|---|---|---|---|---|---|---|---|---|---|
| | | | | | | | LEPR antagonist | | | mAb + αGDF8 + | | |
| | Control mAb | | | αGDF8 + αActA | | | mAb | | | αActA | | |
| | Mean | SEM | N | Mean | SEM | N | Mean | SEM | N | Mean | SEM | N |
| Heart | 0.457 | 0.013 | 11 | 0.473 | 0.005 | 11 | 0.480 | 0.011 | 11 | 0.480 | 0.010 | 12 |
| Liver | 4.576 | 0.140 | 10 | 4.527 | 0.158 | 11 | 5.310 | 0.227 | 11 | 5.170 | 0.159 | 12 |
| Quad | 0.641 | 0.010 | 11 | 0.857 | 0.025 | 11 | 0.663 | 0.019 | 11 | 0.830 | 0.026 | 12 |
| TA Muscle | 0.161 | 0.005 | 11 | 0.206 | 0.005 | 11 | 0.162 | 0.005 | 11 | 0.212 | 0.005 | 12 |
| GA Muscle | 0.512 | 0.013 | 11 | 0.670 | 0.014 | 11 | 0.523 | 0.011 | 11 | 0.672 | 0.015 | 12 |
| Inguinal WAT | 1.590 | 0.169 | 11 | 1.341 | 0.155 | 11 | 3.130 | 0.253 | 11 | 2.826 | 0.224 | 12 |
| Gonadal WAT | 2.392 | 0.231 | 11 | 2.029 | 0.172 | 11 | 5.015 | 0.238 | 11 | 4.441 | 0.201 | 12 |
| BAT | 0.322 | 0.027 | 11 | 0.308 | 0.017 | 11 | 0.583 | 0.045 | 11 | 0.460 | 0.022 | 12 |

TABLE 7
Organ Mass, Muscle Mass, Fat Mass (% Change from Control)

| | | | | | | | | | | LEPR antagonist | | |
|---|---|---|---|---|---|---|---|---|---|---|---|---|
| | | | | | | | LEPR antagonist | | | mAb + αGDF8 + | | |
| | Control mAb | | | αGDF8 + αActA | | | mAb | | | αActA | | |
| | Mean | SEM | N | Mean | SEM | N | Mean | SEM | N | Mean | SEM | N |
| Heart | −3.182E−08 | 2.747 | 11 | 3.480 | 1.151 | 11 | 5.058 | 2.353 | 11 | 5.065 | 2.275 | 12 |
| Liver | −0.00000005 | 3.050 | 10 | −1.055 | 3.460 | 11 | 16.050 | 4.962 | 11 | 12.990 | 3.476 | 12 |
| Quad | −4.545E−09 | 1.520 | 11 | 33.670 | 3.827 | 11 | 3.426 | 2.984 | 11 | 29.490 | 4.132 | 12 |
| TA Muscle | −5.818E−08 | 2.851 | 11 | 27.880 | 2.986 | 11 | 0.460 | 3.097 | 11 | 31.610 | 3.035 | 12 |
| GA Muscle | −4.182E−08 | 2.595 | 11 | 30.940 | 2.757 | 11 | 2.073 | 2.241 | 11 | 31.250 | 2.866 | 12 |
| Inguinal | 6.364E−08 | 10.660 | 11 | −15.660 | 9.761 | 11 | 96.900 | 15.940 | 11 | 77.740 | 14.100 | 12 |
| WAT | | | | | | | | | | | | |
| Gonadal | −2.273E−08 | 9.652 | 11 | −15.170 | 7.197 | 11 | 109.600 | 9.929 | 11 | 85.620 | 8.399 | 12 |
| WAT | | | | | | | | | | | | |
| BAT | 2.455E−08 | 8.318 | 11 | −4.226 | 5.146 | 11 | 81.030 | 13.930 | 11 | 42.800 | 6.818 | 12 |

### Example 3: Combination Treatment of Anti-GDF8 mAb (REGN1033), Anti-Activin a mAb (REGN2477), and LEPR Antagonist mAb (H4H18457P2) in 12 to 14 Week Old Male Mice (+Additional Treatment Groups)

The effects of the specific antagonist anti-LEPR antibody, H4H18457P2, in combination with the anti-MSTN (also referred to as anti-GDF8) and anti-INHBA (also referred to as anti-Activin A) blocking antibodies, H4H1657N2 (REGN1033) and H4H10446P2 (REGN2477), respectively, of the invention, on food intake, body weight, body composition, individual tissue weights, and ex vivo muscle force generation were determined in singly-housed genetically engineered 12 to 14 week old male LEPR^{Hu/Hu} mice, that express a leptin receptor which is composed of the human LEPR ectodomain sequence in place of the murine LEPR ectodomain sequence.

Baseline daily food intake was measured between days −8 and 0. On day −1, baseline whole body lean and fat mass was quantified by NMR. On day 0, mice were stratified to six groups of 7 to 8 mice based on body composition from day −1 and body weight from day 0. Starting on day 0, each group received the respective antibody treatment dose via subcutaneous injection. REGN1033, REGN2477, and the respective IgG4^{P} mAbs are dosed twice a week at 10 mg/kg. H4H18457P2 and its respective IgG4^{P} mAb are dosed once a week at 30 mg/kg. The isotype control (IgG4^{P}) antibody (REGN1945) does not bind any known mouse protein.

### Treatment Groups

$$\\ \begin{matrix}IgG 4 P Control ( \begin{matrix}10 mg / kg + 10 mg / kg , 2 x / week ; \\ 30 mg / kg , 1 x / week\end{matrix} ) , N = 7 & a )\end{matrix}$$

$$\\ \begin{matrix}REGN 1033 ( 10 mg / kg , 2 x / week ) , N = 7 & b )\end{matrix}$$

$$\\ \begin{matrix}REGN 1033 + REGN 2477 ( 10 mg / kg + 10 mg / kg , 2 x / week ) , N = 7 & c )\end{matrix}$$

$$\\ \begin{matrix}H 4 H 18457 P 2 ( 30 mg / kg , 1 x / week ) , N = 7 & d )\end{matrix}$$

$$\\ \begin{matrix}REGN 1033 + H 4 H 18457 P 2 ( 10 mg / kg , 2 x / week ; 30 mg / kg , 1 x / week ) , N = 8 & e )\end{matrix}$$

$$\\ \begin{matrix}REGN 1033 + REGN 2477 + H 4 H 18457 P 2 ( 10 mg / kg + 10 mg / kg , 2 x / week ; 30 mg / kg , 1 x / week ) , N = 8 & f )\end{matrix}$$

Food intake (Table 8B) and body weight (Table 8A) were measured for the duration of the study for each animal. Body composition was quantified on days 6, 13 and 20 (Tables 8C and 8D). On day 21 or 22, animals were euthanized for additional analyses including skeletal muscle fiber number and cross-sectional fiber area (Tables 9-11).

Compared to isotype control antibody, the LEPR antagonist, H4H18457P2, significantly increased body weight and cumulative food intake when administered alone or in combination with REGN1033 or REGN1033 and REGN2477. Mice treated with H4H18457P2 showed significant increases in body weight and cumulative food intake (Tables 8A and 8B) starting at days 13 and 8, respectively, and at subsequent time points, when compared to mice administered isotype control antibody. Body weight and cumulative food intake was significantly elevated with H4H18457P2 and REGN1033 treatment starting at day 7 and at subsequent time points, as compared to isotype control antibody administrations. Mice treated with H4H18457P2, REGN1033 and REGN2477 also showed increased body weight and food intake from days 8 and at 7, respectively, to the end of the study when compared to mice administered isotype control antibody. Mice treated with REGN1033 or REGN1033 and REGN2477 did not exhibit significant changes in body weight or cumulative food intake relative to mice administered control antibody.

Mice treated with H4H18457P2 alone or in combination with REGN1033 or REGN1033 and REGN2477, showed increases in fat mass from baseline at all measured timepoints (days 6, 13 and 20) when compared to mice administered isotype control antibody (Table 8D). At days 13 and 20, mice treated with H4H18457P2, REGN1033 and REGN2477 showed less fat mass gain from baseline than mice treated with H4H18457P2 alone or in combination with REGN1033 (Table 8D). Mice treated with H4H18457P2, REGN1033 and REGN2477 also showed significant increase in lean mass gain from baseline at days 13 and 20 when compared to mice administered isotype control antibody (Table 8C).

Histological analyses revealed no significant effects of any treatment group compared to isotype control antibody administration on the number of muscle fibers in the tibialis anterior or gastrocnemius (Tables 9 and 10). Compared to isotype control antibody delivery, mice treated with REGN1033 in combination with REGN2477 or H4H18457P2 showed increased muscle fiber numbers in the soleus muscle (Table 11). In all three muscles (tibialis anterior, gastrocnemius and soleus) examined, H4H18457P2 treatment did not affect muscle fiber area when compared to isotype control antibody administration (Tables 9, 10 and 11). Mice treated with either REGN1033, REGN1033 and REGN2477, REGN1033 and H4H18457P2, or REGN1033 and REGN2477 and H4H18457P2 showed increased muscle fiber area in the tibialis anterior and gastrocnemius (Tables 9 and 10) when compared to mice administered isotype control antibody. In soleus muscle, only mice treated with the triple combination of H4H18457P2, REGN1033 and REN2477 showed increased muscle fiber area relative to mice administered isotype control antibody (Table 11).

In summary, LEPR antagonist antibody treatment alone increases body weight, food intake and adiposity, and in combination with anti-ActA and/or anti-MSTN blocking antibodies induces additional increases in muscle fiber area or number, respectively.

TABLE 8A
Body Weight (g)

| | | | | | | | | | | | | | | | | REGN1033 + | | |
|---|---|---|---|---|---|---|---|---|---|---|---|---|---|---|---|---|---|---|
| | | | | | | | REGN1033 + | | | | | | REGN1033 + | | | REGN2477 + | | |
| | IgG4^{P} | | | REGN1033 | | | REGN2477 | | | H4H18457P2 | | | H4H18457P2 | | | H4H18457P2 | | |
| | Mean | SEM | N | Mean | SEM | N | Mean | SEM | N | Mean | SEM | N | Mean | SEM | N | Mean | SEM | N |
| Day 0 | 27.267 | 1.145 | 7 | 27.263 | 1.156 | 7 | 27.357 | 1.020 | 7 | 27.650 | 0.803 | 7 | 27.185 | 0.908 | 8 | 27.148 | 1.065 | 8 |
| Day 2 | 27.580 | 1.086 | 7 | 27.927 | 1.161 | 7 | 28.121 | 1.041 | 7 | 29.437 | 0.775 | 7 | 29.141 | 0.849 | 8 | 28.915 | 1.081 | 8 |
| Day 3 | 27.517 | 1.098 | 7 | 28.093 | 1.193 | 7 | 28.094 | 1.023 | 7 | 29.899 | 0.806 | 7 | 29.966 | 0.970 | 8 | 29.650 | 1.081 | 8 |
| Day 6 | 27.464 | 1.090 | 7 | 28.690 | 1.232 | 7 | 28.907 | 1.014 | 7 | 31.124 | 0.934 | 7 | 31.813 | 0.995 | 8 | 31.539 | 1.100 | 8 |
| Day 7 | 27.423 | 1.077 | 7 | 28.611 | 1.215 | 7 | 28.854 | 0.984 | 7 | 31.044 | 0.949 | 7 | 32.003 | 1.025 | 8 | 31.774 | 1.111 | 8 |
| Day 8 | 27.696 | 1.176 | 7 | 28.961 | 1.133 | 7 | 29.186 | 1.076 | 7 | 31.704 | 0.896 | 7 | 32.719 | 1.009 | 8 | 32.340 | 1.136 | 8 |
| Day 9 | 27.723 | 1.152 | 7 | 29.446 | 1.321 | 7 | 29.391 | 1.004 | 7 | 31.963 | 1.015 | 7 | 33.186 | 0.925 | 8 | 32.908 | 1.133 | 8 |
| Day 10 | 27.759 | 1.156 | 7 | 29.169 | 1.144 | 7 | 29.373 | 1.008 | 7 | 32.306 | 0.961 | 7 | 33.569 | 0.966 | 8 | 33.299 | 1.211 | 8 |
| Day 13 | 27.867 | 1.140 | 7 | 29.591 | 1.185 | 7 | 29.831 | 0.933 | 7 | 33.291 | 0.880 | 7 | 34.555 | 1.109 | 8 | 34.550 | 1.308 | 8 |
| Day 14 | 27.883 | 1.162 | 7 | 29.343 | 1.195 | 7 | 29.770 | 0.939 | 7 | 33.059 | 1.026 | 7 | 34.668 | 1.157 | 8 | 34.658 | 1.248 | 8 |
| Day 16 | 28.104 | 1.130 | 7 | 30.213 | 1.186 | 7 | 30.509 | 0.964 | 7 | 33.691 | 1.023 | 7 | 35.610 | 1.142 | 8 | 36.058 | 1.360 | 8 |
| Day 17 | 28.217 | 1.074 | 7 | 29.861 | 1.191 | 7 | 30.259 | 0.978 | 7 | 33.771 | 1.052 | 7 | 35.796 | 1.143 | 8 | 36.114 | 1.347 | 8 |
| Day 20 | 28.304 | 1.167 | 7 | 30.234 | 1.257 | 7 | 30.530 | 0.793 | 7 | 34.724 | 1.118 | 7 | 36.529 | 1.224 | 8 | 36.976 | 1.351 | 8 |
| Day 21 | 28.229 | 1.197 | 7 | 30.264 | 1.242 | 7 | 30.811 | 0.978 | 7 | 34.516 | 1.115 | 7 | 36.604 | 1.258 | 8 | 36.830 | 1.368 | 8 |

TABLE 8B
Cumulative Food Intake

| | | | | | | | | | | | | | | | | REGN1033 + | | |
|---|---|---|---|---|---|---|---|---|---|---|---|---|---|---|---|---|---|---|
| | | | | | | | REGN1033 + | | | | | | REGN1033 + | | | REGN2477 + | | |
| | IgG4^{P} | | | REGN1033 | | | REGN2477 | | | H4H18457P2 | | | H4H18457P2 | | | H4H18457P2 | | |
| | Mean | SEM | N | Mean | SEM | N | Mean | SEM | N | Mean | SEM | N | Mean | SEM | N | Mean | SEM | N |
| Day 0 | 0.000 | 0.000 | 7 | 0.000 | 0.000 | 7 | 0.000 | 0.000 | 7 | 0.000 | 0.000 | 6 | 0.000 | 0.000 | 7 | 0.000 | 0.000 | 8 |
| Day 2 | 9.050 | 0.379 | 7 | 8.993 | 0.601 | 7 | 9.070 | 0.284 | 7 | 10.548 | 0.407 | 6 | 10.527 | 0.400 | 7 | 10.433 | 0.298 | 8 |
| Day 3 | 13.421 | 0.639 | 7 | 13.491 | 0.753 | 7 | 13.533 | 0.434 | 7 | 16.620 | 0.637 | 6 | 16.636 | 0.564 | 7 | 16.754 | 0.477 | 8 |
| Day 6 | 26.191 | 1.373 | 7 | 26.673 | 1.326 | 7 | 26.543 | 0.839 | 7 | 32.998 | 1.303 | 6 | 34.169 | 1.278 | 7 | 34.114 | 0.605 | 8 |
| Day 7 | 30.711 | 1.500 | 7 | 31.011 | 1.476 | 7 | 30.684 | 1.036 | 7 | 38.523 | 1.498 | 6 | 39.857 | 1.364 | 7 | 39.715 | 0.799 | 8 |
| Day 8 | 34.877 | 1.689 | 7 | 35.223 | 1.665 | 7 | 34.610 | 1.258 | 7 | 43.735 | 1.580 | 6 | 45.370 | 1.504 | 7 | 45.015 | 0.918 | 8 |
| Day 9 | 39.234 | 1.955 | 7 | 39.561 | 1.792 | 7 | 38.820 | 1.401 | 7 | 48.983 | 1.831 | 6 | 50.886 | 1.591 | 7 | 50.593 | 1.107 | 8 |
| Day 10 | 43.473 | 2.260 | 7 | 43.599 | 1.934 | 7 | 42.959 | 1.542 | 7 | 54.487 | 1.946 | 6 | 56.586 | 1.615 | 7 | 56.254 | 1.246 | 8 |
| Day 13 | 55.691 | 2.936 | 7 | 56.084 | 2.509 | 7 | 55.276 | 1.921 | 7 | 70.253 | 2.310 | 6 | 73.181 | 1.959 | 7 | 72.801 | 1.886 | 8 |
| Day 14 | 60.170 | 3.083 | 7 | 60.543 | 2.776 | 7 | 59.541 | 1.966 | 7 | 75.147 | 2.497 | 6 | 78.610 | 2.026 | 7 | 78.475 | 2.036 | 8 |
| Day 16 | 68.284 | 3.526 | 7 | 68.890 | 3.181 | 7 | 68.131 | 2.319 | 7 | 84.507 | 2.676 | 6 | 88.901 | 2.155 | 7 | 89.314 | 2.248 | 8 |
| Day 17 | 72.526 | 3.721 | 7 | 72.721 | 3.367 | 7 | 72.154 | 2.450 | 7 | 89.543 | 2.855 | 6 | 93.999 | 2.203 | 7 | 94.939 | 2.429 | 8 |
| Day 20 | 84.754 | 4.432 | 7 | 85.340 | 3.774 | 7 | 85.024 | 2.630 | 7 | 105.160 | 3.373 | 6 | 109.866 | 2.730 | 7 | 111.293 | 2.916 | 8 |
| Day 21 | 88.433 | 4.661 | 7 | 89.089 | 3.962 | 7 | 88.910 | 2.770 | 7 | 109.127 | 3.478 | 6 | 114.414 | 2.916 | 7 | 116.163 | 3.025 | 8 |

TABLE 8C
Lean Mass (g Difference from Baseline)

| | | | | | | | | | | | | | | | | REGN1033 + | | |
|---|---|---|---|---|---|---|---|---|---|---|---|---|---|---|---|---|---|---|
| | | | | | | | REGN1033 + | | | | | | REGN1033 + | | | REGN2477 + | | |
| | IgG4^{P} | | | REGN1033 | | | REGN2477 | | | H4H18457P2 | | | H4H18457P2 | | | H4H18457P2 | | |
| | Mean | SEM | N | Mean | SEM | N | Mean | SEM | N | Mean | SEM | N | Mean | SEM | N | Mean | SEM | N |
| Day −1 | 0.000 | 0.000 | 7 | 0.000 | 0.000 | 7 | 0.000 | 0.000 | 7 | 0.000 | 0.000 | 7 | 0.000 | 0.000 | 8 | 0.000 | 0.000 | 8 |
| Day 6 | −0.026 | 0.107 | 7 | 1.321 | 0.119 | 7 | 1.704 | 0.088 | 7 | 1.227 | 0.242 | 7 | 2.288 | 0.123 | 8 | 2.576 | 0.171 | 8 |
| Day 13 | −0.016 | 0.210 | 7 | 1.930 | 0.151 | 7 | 2.997 | 0.108 | 7 | 1.490 | 0.227 | 7 | 3.201 | 0.197 | 8 | 4.281 | 0.282 | 8 |
| Day 20 | 0.274 | 0.299 | 7 | 2.573 | 0.188 | 7 | 3.873 | 0.336 | 7 | 1.777 | 0.388 | 7 | 3.899 | 0.268 | 8 | 5.716 | 0.328 | 8 |

TABLE 8D
Fat Mass (g Difference from Baseline)

| | | | | | | | | | | | | | | | | REGN1033 + | | |
|---|---|---|---|---|---|---|---|---|---|---|---|---|---|---|---|---|---|---|
| | | | | | | | REGN1033 + | | | | | | REGN1033 + | | | REGN2477 + | | |
| | IgG4^{P} | | | REGN1033 | | | REGN2477 | | | H4H18457P2 | | | H4H18457P2 | | | H4H18457P2 | | |
| | Mean | SEM | N | Mean | SEM | N | Mean | SEM | N | Mean | SEM | N | Mean | SEM | N | Mean | SEM | N |
| Day −1 | 0 | 0 | 7 | 0 | 0 | 7 | 0 | 0 | 7 | 0 | 0 | 7 | 0 | 0 | 8 | 0 | 0 | 8 |
| Day 6 | −0.027 | 0.084 | 7 | −0.009 | 0.087 | 7 | −0.099 | 0.091 | 7 | 1.909 | 0.170 | 7 | 2.028 | 0.128 | 8 | 1.496 | 0.197 | 8 |
| Day 13 | 0.250 | 0.128 | 7 | 0.273 | 0.120 | 7 | −0.340 | 0.133 | 7 | 3.580 | 0.295 | 7 | 3.800 | 0.222 | 8 | 2.600 | 0.270 | 8 |
| Day 20 | 0.500 | 0.207 | 7 | 0.220 | 0.166 | 7 | −0.319 | 0.180 | 7 | 4.834 | 0.469 | 7 | 5.158 | 0.304 | 8 | 3.614 | 0.485 | 8 |

TABLE 9
TA Muscle Fiber Area and Number

| | | | | | | | | | | | | | | | | REGN1033 + | | |
|---|---|---|---|---|---|---|---|---|---|---|---|---|---|---|---|---|---|---|
| | | | | | | | REGN1033 + | | | | | | REGN1033 + | | | REGN2477 + | | |
| | IgG4^{P} | | | REGN1033 | | | REGN2477 | | | H4H18457P2 | | | H4H18457P2 | | | H4H18457P2 | | |
| | (N = 7) | | | (N = 7) | | | (N = 7) | | | (N = 7) | | | (N = 8) | | | (N = 8) | | |
| | Mean | SEM | N | Mean | SEM | N | Mean | SEM | N | Mean | SEM | N | Mean | SEM | N | Mean | SEM | N |
| Muscle Size | 2004 | 56.01 | 7 | 2535 | 73.66 | 7 | 2841 | 150.5 | 7 | 2316 | 82.99 | 7 | 2491 | 87.98 | 8 | 3011 | 147.6 | 8 |
| (um\hat{ }2) | | | | | | | | | | | | | | | | | | |
| Fiber | 1982 | 117.1 | 7 | 1826 | 94.79 | 7 | 1839 | 115.7 | 7 | 1862 | 124.4 | 7 | 2018 | 163.2 | 8 | 1670 | 106.7 | 8 |
| Number | | | | | | | | | | | | | | | | | | |
| (Number of | | | | | | | | | | | | | | | | | | |
| Fibers) | | | | | | | | | | | | | | | | | | |

TABLE 10
GA Muscle Fiber Area and Number

| | | | | | | | | | | | | | | | | REGN1033 + | | |
|---|---|---|---|---|---|---|---|---|---|---|---|---|---|---|---|---|---|---|
| | | | | | | | REGN1033 + | | | | | | REGN1033 + | | | REGN2477 + | | |
| | Igg4^{P} | | | REGN1033 | | | REGN2477 | | | H4H18457P2 | | | H4H18457P2 | | | H4H18457P2 | | |
| | (N = 7) | | | (N = 7) | | | (N = 7) | | | (N = 7) | | | (N = 8) | | | (N = 8) | | |
| | Mean | SEM | N | Mean | SEM | N | Mean | SEM | N | Mean | SEM | N | Mean | SEM | N | Mean | SEM | N |
| Muscle Size | 1903 | 69.1 | 7 | 2444 | 127.2 | 7 | 2319 | 42.42 | 7 | 1902 | 85.57 | 7 | 2340 | 52.31 | 8 | 2562 | 106.1 | 8 |
| (um\hat{ }2) | | | | | | | | | | | | | | | | | | |
| Fiber Number | 4992 | 510 | 7 | 3864 | 368.7 | 7 | 4615 | 336.5 | 7 | 4830 | 576.7 | 7 | 4189 | 328.5 | 8 | 3748 | 460.1 | 8 |
| (Number Of | | | | | | | | | | | | | | | | | | |
| Fibers) | | | | | | | | | | | | | | | | | | |

TABLE 11
Soleus Muscle Fiber Area and Number

| | | | | | | | | | | | | | | | | REGN1033 + | | |
|---|---|---|---|---|---|---|---|---|---|---|---|---|---|---|---|---|---|---|
| | | | | | | | REGN1033 + | | | | | | REGN1033 + | | | REGN2477 + | | |
| | IgG4^{P} | | | REGN1033 | | | REGN2477 | | | H4H18457P2 | | | H4H18457P2 | | | H4H18457P2 | | |
| | (N = 7) | | | (N = 7) | | | (N = 7) | | | (N = 7) | | | (N = 8) | | | (N = 8) | | |
| | Mean | SEM | N | Mean | SEM | N | Mean | SEM | N | Mean | SEM | N | Mean | SEM | N | Mean | SEM | N |
| Muscle Size | 1253 | 64.74 | 7 | 1590 | 188.8 | 7 | 1377 | 43.75 | 7 | 1235 | 79.26 | 7 | 1455 | 52.5 | 8 | 1696 | 84.62 | 8 |
| (um\hat{ }2) | | | | | | | | | | | | | | | | | | |
| Fiber | 539.4 | 71.96 | 7 | 650.3 | 87.98 | 7 | 797.3 | 34.8 | 7 | 729.9 | 41.15 | 7 | 811.4 | 24.63 | 8 | 726.4 | 54.47 | 8 |
| Number | | | | | | | | | | | | | | | | | | |
| (Number of | | | | | | | | | | | | | | | | | | |
| Fibers) | | | | | | | | | | | | | | | | | | |

### Example 4: Combination Treatment of Anti-GDF8 mAb (REGN1033), Anti-Activin a mAb (REGN2477), and LEPR Antagonist mAb (H4H184572P2) in Male Mice

The effects of the specific antagonist anti-LEPR antibody, H4H18457P2, in combination with the anti-MSTN (also referred to as GDF8) and anti-INHBA (also referred to as Activin A) blocking antibodies, H4H1657N2 (REGN1033) and H4H10446P2 (REGN2477), respectively, on food intake, body weight, body composition, individual tissue weights, and ex vivo muscle force generation were determined in singly-housed genetically engineered 12 to 14 week old male LEPR^{Hu/Hu} mice, that express a leptin receptor which is composed of the human LEPR ectodomain sequence in place of the murine LEPR ectodomain sequence.

Baseline daily food intake was measured between days −8 and 0. On day −1, baseline whole body lean and fat mass was quantified by NMR. On day 0, mice were stratified to six groups of 7 to 8 mice based on body composition from day −1 and body weight from day 0. Starting on day 0, each group received the respective antibody treatment dose via subcutaneous injection. REGN1033, REGN2477, and the respective IgG4^{P} mAbs were dosed twice a week at 10 mg/kg. H4H18457P2 and its respective IgG4^{P} mAb were dosed once a week at 30 mg/kg. The isotype control (IgG4^{P}) antibody does not bind any known mouse protein.

### Treatment Groups

$$\\ \begin{matrix}IgG 4 P Control ( \begin{matrix}10 mg / kg + 10 mg / kg , 2 x / week ; \\ 30 mg / kg , 1 x / week\end{matrix} ) , N = 7 & a )\end{matrix}$$

$$\\ \begin{matrix}REGN 1033 ( 10 mg / kg , 2 x / week ) , N = 7 & b )\end{matrix}$$

$$\\ \begin{matrix}REGN 1033 + REGN 2477 ( 10 mg / kg + 10 mg / kg , 2 x / week ) , N = 7 & c )\end{matrix}$$

$$\\ \begin{matrix}H 4 H 18457 P 2 ( 30 mg / kg , 1 x / week ) , N = 7 & d )\end{matrix}$$

$$\\ \begin{matrix}REGN 1033 + H 4 H 18457 P 2 ( 10 mg / kg , 2 x / week ; 30 mg / kg , 1 x / week ) , N = 8 & e )\end{matrix}$$

$$\\ \begin{matrix}REGN 1033 + REGN 2477 + H 4 H 18457 P 2 ( 10 mg / kg + 10 mg / kg , 2 x / week ; 30 mg / kg , 1 x / week ) , N = 8 & f )\end{matrix}$$

Food intake and body weight were measured for the duration of the study for each animal. Body composition was quantified on days 6, 13 and 20. On day 21 or 22, animals were euthanized, individual organ and skeletal muscle tissues were weighed and collected for additional analyses including liver triglyceride quantification and skeletal muscle fiber number and cross-sectional fiber area.

Quantification of liver triglyceride content revealed that mice treated with H4H18457P2 alone or in combination with REGN1033, showed increased liver triglyceride content when compared to mice administered isotype control antibody (Table 12). In contrast, liver triglyceride content was not increased in mice treated with H4H18457P2 in combination with REGN1033 and REGN2477 when compared to mice administered isotype control antibody (Table 12). Mice treated with H4H18457P2 in combination with REGN1033 and REGN2477 exhibited decreased liver triglyceride content when compared with mice treated with H4H18457P2 in combination with REGN1033 (Table 12). In summary, LEPR antagonist antibody treatment increased liver triglyceride content that is mitigated in a combination treatment with LEPR antagonist, anti-ActA and anti-MSTN blocking antibodies.

TABLE 12
Liver Triglycerides

| | | | | | | | | | | | | | | | | REGN1033 + | | |
|---|---|---|---|---|---|---|---|---|---|---|---|---|---|---|---|---|---|---|
| | | | | | | | REGN1033 + | | | | | | REGN1033 + | | | REGN2477 + | | |
| | IgG4^{P} | | | REGN1033 | | | REGN2477 | | | H4H18457P2 | | | H4H18457P2 | | | H4H18457P2 | | |
| | Mean | SEM | N | Mean | SEM | N | Mean | SEM | N | Mean | SEM | N | Mean | SEM | N | Mean | SEM | N |
| Liver | 8.927 | 6.163 | 7 | 9.687 | 6.552 | 5** | 8.093 | 5.782 | 7 | 19.09 | 8.495 | 6* | 19.18 | 13.53 | 8 | 12.53 | 5.846 | 8 |
| Triglyceride | | | | | | | | | | | | | | | | | | |
| (mg/g wet | | | | | | | | | | | | | | | | | | |
| weight) | | | | | | | | | | | | | | | | | | |

**Data for 2 out of 7 mice not included in values shown
*Data for 1 out of 7 mice not included in values shown

We claim:

1. A combination comprising:
a leptin receptor antagonist in association with
a GDF8 antagonist in association with
an Activin A antagonist; and
optionally, a pharmaceutically acceptable carrier or diluent.

2. The combination of claim 1 wherein at least two of said antagonists selected from leptin receptor antagonist, GDF8 antagonist and Activin A antagonist are co-formulated.

3. The combination of claim 1, wherein said antagonists are in separate compositions.

4. The combination of claim 1, wherein the leptin receptor antagonist, the GDF8 antagonist and/or the Activin A antagonist is an antibody or antigen-binding fragment thereof that binds specifically to leptin receptor, GDF8 and/or Activin A, respectively.

5. The combination of claim 1, wherein the leptin receptor antagonist is an antibody or antigen-binding fragment which specifically binds to the receptor and does not compete with leptin for binding to the receptor.

6. The combination of claim 1, wherein
(i) the leptin receptor antagonist is an antibody or an antigen-binding fragment thereof that specifically binds to leptin receptor comprising:
a heavy chain variable region that comprises CDR-H1, CDR-H2, CDR-H3 of a heavy chain variable region selected from H4H17322P2, H4H18437P2, H4H18439P2, H4H18440P2, H4H18457P2, H4H18462P2, H4H18464P2, H4H18466P2 and H4H18508P2;
and
a light chain variable region that comprises CDR-L1, CDR-L2, CDR-L3 of a light chain variable region selected from H4H17322P2, H4H18437P2, H4H18439P2, H4H18440P2, H4H18457P2, H4H18462P2, H4H18464P2, H4H18466P2 and H4H18508P2,
and/or
that binds to the same epitope on leptin receptor as said antibody or fragment and/or that competes for binding to leptin receptor with said antibody or fragment;
(ii) the GDF8 antagonist is an antibody or an antigen-binding fragment thereof that specifically binds to GDF8 that comprises:
a heavy chain variable region that comprises CDR-H1, CDR-H2, CDR-H3 of a heavy chain variable region selected from 21-E5; 21-B9; 21-E9; 21-A2; 22-D3; 22-E6; 22-G10; 1A2; 20B12; 58C8; 19F2; 8D12-1; 4E3-7; 9B11-12; 4B9; 1H4-5; 9B4-3; 3E2-1; 4G3-25; 4B6-6; H4H1657N2 or H4H1669P and H4H18508P2;
and
a light chain variable region that comprises CDR-L1, CDR-L2, CDR-L3 of a light chain variable region selected from 21-E5; 21-B9; 21-E9; 21-A2; 22-D3; 22-E6; 22-G10; 1A2; 20B12; 58C8; 19F2; 8D12-1; 4E3-7; 9B11-12; 4B9; 1H4-5; 9B4-3; 3E2-1; 4G3-25; 4B6-6; H4H1657N2 or H4H1669P and H4H18508P2,
and/or
that binds to the same epitope on GDF8 as said antibody or fragment and/or that competes for binding to GDF8 with said antibody or fragment; and/or
(iii) the Activin A antagonist is an antibody or an antigen-binding fragment thereof that specifically binds to Activin A that comprises:
a heavy chain variable region that comprises CDR-H1, CDR-H2, CDR-H3 of a heavy chain variable region selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and H2aM10965N
and
a light chain variable region that comprises CDR-L1, CDR-L2, CDR-L3 of a light chain variable region selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and H2aM10965N,
and/or
that binds to the same epitope on Activin A as said antibody or fragment and/or that competes for binding to Activin A with said antibody or fragment.

7. The combination of claim 1, wherein
(i) the LEPR antagonist is an antibody or an antigen-binding fragment thereof comprising:
a heavy chain variable region of an antibody selected from H4H17322P2, H4H18437P2, H4H18439P2, H4H18440P2, H4H18457P2, H4H18462P2, H4H18464P2, H4H18466P2 and H4H18508P2;
and
a light chain variable region of an antibody selected from H4H17322P2, H4H18437P2, H4H18439P2, H4H18440P2, H4H18457P2, H4H18462P2, H4H18464P2, H4H18466P2 and H4H18508P2,
or
that binds to the same epitope on LEPR as said antibody or fragment and/or that competes for binding to LEPR with said antibody or fragment;
(ii) the GDF8 antagonist is an antibody or an antigen-binding fragment thereof comprising:
a heavy chain variable region of an antibody selected from 21-E5; 21-B9; 21-E9; 21-A2; 22-D3; 22-E6; 22-G10; 1A2; 20B12; 58C8; 19F2; 8D12-1; 4E3-7; 9B11-12; 4B9; 1H4-5; 9B4-3; 3E2-1; 4G3-25; 4B6-6; H4H1657N2 or H4H1669P and H4H18508P2;
and
a light chain variable region of an antibody selected from 21-E5; 21-B9; 21-E9; 21-A2; 22-D3; 22-E6; 22-G10; 1A2; 20B12; 58C8; 19F2; 8D12-1; 4E3-7; 9B11-12; 4B9; 1H4-5; 9B4-3; 3E2-1; 4G3-25; 4B6-6; H4H1657N2 or H4H1669P and H4H18508P2,
or
that binds to the same epitope on GDF8 as said antibody or fragment and/or that competes for binding to GDF8 with said antibody or fragment;
and/or
(iii) the Activin A antagonist is an antibody or an antigen-binding fragment thereof comprising:
a heavy chain variable region of an antibody selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and H2aM10965N;
and
a light chain variable region of an antibody selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and H2aM10965N,
or
that binds to the same epitope on Activin A as said antibody or fragment and/or that competes for binding to Activin A with said antibody or fragment.

8. The combination of claim 1, which comprises antibody H4H18457P2, antibody REGN2477 and antibody REGN1033.

9. The combination of claim 1, comprising a further therapeutic agent.

10. The combination of claim 9 wherein the further therapeutic agent is one or more selected from appetite stimulant, a cannabinoid, an angiotensin-converting enzyme (ACE) inhibitor, an angiotensin receptor blocker, a smooth muscle relaxant, a nitrate, a diuretic, iron, a bronchodilator, an anticholinergic, a corticosteroid, an antibiotic, a nonsteroidal anti-inflammatory drug (NSAID), an immunosuppressant, an HMG-CoA reductase inhibitor, an anti-depressant, an anti-cancer therapy or a topical agent.

11. An injection device or vessel comprising the combination of claim 1.

12. The device or vessel of claim 11 which is a vessel which is a vial.

13. The device or vessel of claim 11 which is an injection device which is a hypodermic needle and syringe, an autoinjector or a pre-filled syringe.

14. A method for administering a combination comprising a leptin receptor antagonist in association with a GDF8 antagonist in association with an Activin A antagonist; and
optionally, a pharmaceutically acceptable carrier or diluent; to a subject comprising introducing the components of the combination into the body of the subject.

15. The method of claim 14 wherein the components are administered parenterally.

16. A method for inhibiting LEPR, GDF8 and Activin A in the body of a subject comprising administering, to the subject, a therapeutically effective amount of a combination comprising a leptin receptor antagonist in association with a GDF8 antagonist in association with an Activin A antagonist.

17. A method for increasing food intake, adiposity, body weight, muscle strength, muscle fiber size and/or lean mass, in a subject in need thereof, comprising administering a therapeutically effective amount of a combination comprising a leptin receptor antagonist in association with a GDF8 antagonist in association with an Activin A antagonist to the subject.

18. The method of claim 17 for increasing lean mass wherein said increase is at the expense of fat mass.

19. A method for increasing athletic performance in a subject in need thereof comprising administering a therapeutically effective amount of a combination comprising a leptin receptor antagonist in association with a GDF8 antagonist in association with an Activin A antagonist to the subject.

20. The method of claim 19 wherein the subject is undergoing physical therapy.

21. The method of claim 14, wherein the subject suffers from one or more selected from malnutrition, failure to thrive, insufficient food intake, an eating disorder, cachexia, muscle atrophy or wasting and muscle injury;
and/or,
is undergoing stroke rehabilitation.

22. A method for treating or preventing malnutrition, cachexia, failure to thrive, an eating disorder characterized by inadequate caloric intake, muscle atrophy, age-related sarcopenia or muscle injury, in a subject in need thereof, comprising administering a therapeutically effective amount of a combination comprising a leptin receptor antagonist in association with a GDF8 antagonist in association with an Activin A antagonist to the subject.

23. A method for mitigating increased liver triglyceride content in a subject administered an leptin receptor antagonist comprising administering, to the subject, in association with the leptin receptor antagonist, a GDF8 antagonist in association with an Activin A antagonist.

24. The method of claim 14, wherein:
(i) the LEPR antagonist is an antibody or an antigen-binding fragment thereof that specifically binds to LEPR comprising:
a heavy chain variable region that comprises CDR-H1, CDR-H2, CDR-H3 of a heavy chain variable region selected from H4H17322P2, H4H18437P2, H4H18439P2, H4H18440P2, H4H18457P2, H4H18462P2, H4H18464P2, H4H18466P2 and H4H18508P2;
and
a light chain variable region that comprises CDR-L1, CDR-L2, CDR-L3 of a light chain variable region selected from H4H17322P2, H4H18437P2, H4H18439P2, H4H18440P2, H4H18457P2, H4H18462P2, H4H18464P2, H4H18466P2 and H4H18508P2,
and/or
that binds to the same epitope on LEPR as said antibody or fragment and/or that competes for binding to LEPR with said antibody or fragment;
(ii) the GDF8 antagonist is an antibody or an antigen-binding fragment thereof that specifically binds to GDF8 comprising:
a heavy chain variable region that comprises CDR-H1, CDR-H2, CDR-H3 of a heavy chain variable region selected from 21-E5; 21-B9; 21-E9; 21-A2; 22-D3; 22-E6; 22-G10; 1A2; 20B12; 58C8; 19F2; 8D12-1; 4E3-7; 9B11-12; 4B9; 1H4-5; 9B4-3; 3E2-1; 4G3-25; 4B6-6; H4H1657N2 or H4H1669P and H4H18508P2;
and
a light chain variable region that comprises CDR-L1, CDR-L2, CDR-L3 of a light chain variable region selected from 21-E5; 21-B9; 21-E9; 21-A2; 22-D3; 22-E6; 22-G10; 1A2; 20B12; 58C8; 19F2; 8D12-1; 4E3-7; 9B11-12; 4B9; 1H4-5; 9B4-3; 3E2-1; 4G3-25; 4B6-6; H4H1657N2 or H4H1669P and H4H18508P2,
and/or
that binds to the same epitope on GDF8 as said antibody or fragment and/or that competes for binding to GDF8 with said antibody or fragment; and/or
(iii) the Activin A antagonist is an antibody or an antigen-binding fragment thereof that specifically binds to Activin A comprising:
a heavy chain variable region that comprises CDR-H1, CDR-H2, CDR-H3 of a heavy chain variable region selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and H2aM10965N
and
a light chain variable region that comprises CDR-L1, CDR-L2, CDR-L3 of a light chain variable region selected from H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P22, H4H10447P2, H4H10448P2, H4H10452P2, H4H10468P2 and H2aM10965N,
and/or
that binds to the same epitope on Activin A as said antibody or fragment and/or that competes for binding to Activin A with said antibody or fragment.

25. A method for making a combination of claim 1, comprising co-formulating the LEPR antagonist, the GDF8 antagonist and the Activin A antagonist and, optionally, a pharmaceutically acceptable carrier.

26. A combination which is the product of the method of claim 25.

27. A method of making the device or vessel of claim 11, comprising introducing the components of the combination into the vessel or device.

28. A device or vessel which is the product of the method of claim 27.
